(19) United States
(12) Patent Application Publication
(10) Pub. No.: US 2024/0350043 A1
(43) Pub. Date: Oct. 24, 2024

(54) USE OF FREQUENCY DIVISION MULTIPLEXING FOR OPTICAL CARDIAC SIGNALS

(71) Applicant: Garmin International, Inc., Olathe, KS (US)

(72) Inventors: Paul R. MacDonald, Cochrane (CA); James K. Rooney, Cochrane (CA); Tim A. Verschaeve, Cochrane (CA)

(21) Appl. No.: 18/637,928

(22) Filed: Apr. 17, 2024

Related U.S. Application Data
(60) Provisional application No. 63/635,063, filed on Apr. 17, 2024.
(60) Provisional application No. 63/496,884, filed on Apr. 18, 2023.

(51) Int. Cl. A61B 5/1455; A61B 5/00

(52) U.S. Cl. CPC A61B 5/14552; A61B 5/681; A61B 5/7228; A61B 5/725; A61B 2562/0238

(57) ABSTRACT

An electronic wearable device comprises a housing configured to contact a user's skin, first and second modulators, first and second optical transmitters coupled with the first and second modulators, an optical receiver, and receive processing circuits. The first and second modulators output a first electronic signal having a first carrier frequency and a second electronic signal having a second carrier frequency. The first optical transmitter outputs a first optical signal having the first carrier frequency into the user's skin. The second optical transmitter outputs a second optical signal having the second carrier frequency into the user's skin. The optical receiver receives the optical signals from the skin as a broadband signal. The receive processor circuits output a first photoplethysmogram signal corresponding to the first optical signal based on the first carrier frequency and a second photoplethysmogram signal corresponding to the second optical signal based on the second carrier frequency.

### RELATED APPLICATIONS

The current patent application is a non-provisional utility patent application which claims priority benefit, with regard to all common subject matter, under 35 U.S.C. § 119 (c) of earlier-filed U.S. Provisional Application Ser. No. 63/496,884, filed Apr. 18, 2023, and entitled “MULTIPLEXING OF MODULATED OPTICAL SIGNALS,” and U.S. Provisional Application Ser. No. 63/635,063, filed Apr. 17, 2024, and entitled “ANALOG FRONT END CONFIGURED TO USE FREQUENCY DIVISION MULTIPLEXING FOR OPTICAL CARDIAC SIGNALS,” The Provisional Applications are hereby incorporated by reference, in their entirety, into the current patent application.

### BACKGROUND

An electronic wearable device may provide optical cardiac monitoring of a user of the device. The user may wear the electronic device such that a housing of the electronic device is located in contact with the skin of the user-typically being worn on the user's wrist. The cardiac monitoring may include physiological metrics and information such as a user's heart rate and pulse oximetry.

Many conventional electronic wearable devices are body-worn devices include a housing having a bottom wall containing optical transmitters (e.g., LEDs) that output (emit) optical signals (light) into a user's extremity, such as the user's wrist, and optical receivers (photodiodes) that receive reflections of the emitted light. The conventional devices typically include one or more processors that generate photoplethysmogram (PPG) signals associated with the intensity of reflected light and use the generated PPG signals to determine physiological information about the user, including cardiac metrics, such as a user's heart rate or and blood oxygen saturation. A transmitter array may form a plurality of optical transmitters, some of which emit light at a different wavelength than the light emitted by other optical transmitters. It is common for conventional devices to incorporate a plurality of transmitter arrays and a plurality of optical receivers at different locations on the bottom wall of the housing, which enables a plurality of optical signals to pass through different paths, in some cases, using different wavelengths of optical signals. The techniques used to control the output of optical signals and positioning of optical components may impact the quality of reflections received by the optical sensor(s) and the corresponding PPG signal, which likely impacts the accuracy of measurements and determinations of cardiac information about the user. Many optical layouts and topologies are known.

Conventional electronic wearable devices typically utilize time division multiplexing to output light from the different locations to one or more optical receivers without simultaneous emissions by optical transmitters that are proximate to each other as such time division multiplexing techniques typically result in the emission of light by only one optical transmitter (proximate to the one or more optical receivers) at any given moment. Each optical transmitter is typically allocated a certain time slot to emit its optical signals and a plurality of optical transmitters sequentially output a plurality of optical signals over a larger period of time. For instance, time slots of five milliseconds (msec) may be allocated to four optical transmitters to transmit optical signals over a period of twenty msec, which would result in four sequential optical transmissions from the four optical transmitters in twenty msec. Typically, the time slots for optical transmitters that are proximate to one another do not overlap, which results in each optical transmitter emitting its optical signals into a region of the user's wrist during a period of time that is allocated to that optical transmitter (i.e., two optical transmitters that are proximate to one another do not emit light simultaneously). Accordingly, a PPG signal generated based on an electronic signal output by an optical receiver (corresponding to the intensity of light reflected from the user's skin) has corresponding time slots that are each associated with one optical transmitter that emitted an optical signal. The optical transmitters of the conventional electronic wearable devices that implement a time division multiplexing technique do not output optical signals simultaneously and continuously.

Referring to FIG. 1A, the conventional electronic wearable device may include optical devices, such as optical transmitters that emit an optical signal into the user's skin and optical receivers that receive transmissions or reflections of the optical signal from the skin and generate a photoplethysmogram (PPG) signal corresponding to the intensity of the received optical signal. Typically, each optical transmitter emits the optical signal at a constant optical intensity. Having multiple optical transmitters each emitting an optical signal within one or more predetermined timeslots provides greater accuracy and additional functionality as each optical receiver receives optical signals from all optical transmitters having a signal path to the respective optical receiver and generates a PPG electronic signal. Accordingly, a signal processor may utilize the one or more predetermined timeslots for each optical transmitter to identify the source (an optical transmitter) of each PPG electronic signal generated by each optical transmitter.

As shown in FIG. 1B, in order for a signal processor to associate each PPG electronic signal generated by an optical receiver with an optical transmitter, the signal processor typically retrieves from a memory element one or more time periods (or time slots) during which each optical transmitter emits its optical signal, as shown in FIG. 1B. For example, a signal processor may configure a first optical transmitter (TX1) to output (emit) its optical signal during a first time window (period) before turning off, a second optical transmitter (TX2) to output (emit) its optical signal during a second time window (period) before turning off, and so forth successively, until the Nth optical transmitter (TXN) has transmitted its optical signal during an Nth time window (period), at which point, the process may repeat as long as desired. As the signal processor determines which optical transmitter was configured to transmit its optical signal during any time period (the memory may store such information), the signal processor can determine the source of the optical signal and the signal path from the optical transmitter to the optical receiver. This conventional technique for utilizing a plurality of optical transmitters is an example application of time division multiplexing (TDM) optical signal transmission.

Although time division multiplexing enables sequential emissions from a plurality of optical transmitters, the time period required for all of the optical transmitters to be allocated a timeslot increases with the number of optical transmitters. As a result, the time period required for a plurality of optical transmitters to emit their optical signals (each during its allocated time slot) may limit the rate at which one or more processors can determine information using PPG signals generated using reflections of the optical signals from a user's wrist. The duration of each emission may also be correlated to a desired signal-to-noise ratio of the PPG signal, which may require each emission of optical signals to be of a minimum duration for accurate determinations of physiological information about the user.

Additionally, with emergence of improved optical technologies for determining physiological information of a user, there is an increased desire for the plurality of optical signals to output optical signals at wavelengths (of light) within an electromagnetic wave spectrum that includes a visible range (e.g., 520 nm wavelength or light that visually appears “green”) and an infrared range (that is not visible to the human eye) to produce PPG signals that are best-suited to determine physiological information about the user. A known application of multiwavelength PPG is pulse spectrometry, which includes pulse oximetry. Pulse oximetry typically requires generating PPG signals using optical signals that were output at different wavelengths, such as a first wavelength between 600-700 nm and a second wavelength between 900-1000 nm. Naturally, as the number of optical transmitters increases, the time period over which each PPG signal is generated is reduced.

Accordingly, conventional time division multiplexing techniques limit the number of optical signals that may be output by the optical transmitters, independently or within an optical transmitter array, to a sequence of optical transmissions that commonly results in one optical signal traveling along a signal path to an optical receiver in an area of the user's skin for each time period, where a separate time window exists for each transmission and/or a separation of areas of the user's wrist through which optical signals through (along signal paths between optical transmitters and optical receivers) for each time period.

### SUMMARY

Embodiments of the present technology provide an improved electronic wearable device comprising, inter alia, a plurality of optical transmitters, each of which is configured to emit an optical signal having a predetermined carrier frequency and a signal processor configured to control each optical transmitter to utilize a predetermined carrier frequency, thereby enabling frequency division multiplexing optical signal transmission, which allows one or more (or all) of the optical transmitters to emit the optical signals simultaneously and continuously. Such enhancements enable improved accuracy in determining physiological information such as a user's heart rate, pulse oximetry and other parameters. Putting aside power consumption and other considerations, a signal processor of the improved electronic wearable device may control a plurality of optical transmitters to each output an optical signal simultaneously, utilize a stored predetermined carrier frequency to determine the optical transmitter from which each optical signal corresponding to a PPG electronic signal was output and a signal path along which the optical signal traveled to an optical receiver, select a signal path by determining which PPG electronic signal corresponds to the selected signal path and determine information about the user based on the determined PPG electronic signal corresponding to the selected signal path.

An exemplary electronic wearable device may broadly comprise a housing including a bottom wall configured to contact a user's skin, a first modulator, a second modulator, a first optical transmitter, a second optical transmitter, an optical receiver, a first receive processor circuit, and a second receive processor circuit. The first modulator is configured to output a first modulated electronic signal having a first carrier frequency. The second modulator is configured to output a second modulated electronic signal having a second carrier frequency. The first optical transmitter is coupled with the first modulator, positioned on the bottom wall and configured to output a first optical signal into a user's skin, wherein the first optical signal has the first carrier frequency. The second optical transmitter is coupled with the second modulator, positioned on the bottom wall and configured to output a second optical signal into a user's skin, wherein the second optical signal has the second carrier frequency. The optical receiver is positioned on the bottom wall and configured to receive the first and second optical signals from the user's skin as a broadband electronic signal including the first optical signal and the second optical signal associated with the first carrier frequency and the second carrier frequency, respectively, and output the broadband electronic signal. The first receive processor circuit is configured to receive the broadband electronic signal and output a first photoplethysmogram (PPG) electronic signal corresponding to the first optical signal based on the first carrier frequency. The second receive processor circuit is configured to receive the broadband electronic signal and output a second PPG electronic signal corresponding to the second optical signal based on the second carrier frequency.

Another exemplary electronic wearable device may broadly comprise a housing including a bottom wall configured to contact a user's skin, a first modulator, a second modulator, a first optical transmitter, a second optical transmitter, an optical receiver, an analog to digital converter, a memory clement and a processor. The first modulator is configured to output a first modulated electronic signal having a first carrier frequency. The second modulator is configured to output a second modulated electronic signal having a second carrier frequency. The first optical transmitter is coupled with the first modulator, positioned on the bottom wall and configured to output a first optical signal into a user's skin, wherein the first optical signal has the first carrier frequency. The second optical transmitter is coupled with the second modulator, positioned on the bottom wall and configured to output a second optical signal into a user's skin, wherein the second optical signal has the second carrier frequency. The optical receiver is positioned on the bottom wall and configured to receive the first and second optical signals from the user's skin as a broadband electronic signal including the first optical signal and the second optical signal associated with the first carrier frequency and the second carrier frequency, respectively, and output the broadband electronic signal. The analog to digital converter is configured to receive the analog broadband electronic signal and output the analog broadband electronic signal as a stream of digital data values corresponding to the first optical signal and the second optical signal. The memory element is configured to store the first carrier frequency as corresponding to the first optical signal and the second carrier frequency as corresponding to the second optical signal. The processor is configured to receive the digital broadband electronic signal including the first optical signal having the first carrier frequency and the second optical signal having the second carrier frequency, generate a first filtered electronic signal and a second filtered electronic signal having the first carrier frequency and the second carrier frequency, respectively, demodulate the first filtered electronic signal and the second filtered electronic signal by removing the first carrier frequency and the first carrier frequency, respectively, and output a first PPG electronic signal and a second PPG electronic signal corresponding to the demodulated first filtered electronic signal and the demodulated second filtered electronic signal, respectively.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present technology will be apparent from the following detailed description of the embodiments and the accompanying drawing figures.

### BRIEF DESCRIPTION OF DRAWINGS

Embodiments of the present technology are described in detail below with reference to the attached drawing figures, wherein:

FIG. 1A is a schematic block diagram of a prior art circuit of a plurality of optical transmitters outputting optical signals that are received by a plurality of optical receivers of a wrist-worn electronic device and used to generate a photoplethysmogram (PPG) signal;

FIG. 1B is a plot of a plurality of optical transmissions occurring over a period of time to implement a time division multiplexing transmission scheme for the prior art circuit of FIG. 1A;

FIG. 2 is a top view of an electronic wearable device constructed in accordance with various embodiments of the present technology, the electronic wearable device being worn on a user's wrist;

FIG. 3 is a bottom view of the electronic wearable device, illustrating a plurality of optical transmitters and a plurality of optical receivers on a bottom wall of a housing of the electronic wearable device;

FIG. 4 is a cross sectional view of the electronic wearable device cut along a vertical plane, illustrating an optical transmitter emitting an optical signal through and/or reflected by the user's skin and received by an optical receiver;

FIG. 5 is a schematic block diagram of various electronic components of the electronic wearable device;

FIG. 6A is a schematic block diagram of a plurality of circuits and devices of the electronic wearable device that implement a transmit signal flow illustrating a plurality of modulators and a plurality of optical transmitters outputting optical signals into the user's skin that are received by at least one of a plurality of optical receivers;

FIG. 6B is a schematic block diagram of a plurality of circuits and devices of the electronic wearable device that implement a receive signal flow illustrating a plurality of receive processor circuits filtering and demodulating optical signals that one or more optical receivers received from the user's skin to generate PPG electronic signals;

FIG. 7 is a plot of amplitude for a plurality of filter carrier frequencies;

FIG. 8 is a schematic block diagram of a plurality of optical transmitters and an optical receiver that implement a transmit signal flow and a receive signal flow;

FIG. 9A is a schematic block diagram of a plurality of optical transmitter arrays and a plurality of optical receivers illustrating pathways of the optical signal transmitted by each of the optical transmitters and received by a plurality of optical receivers;

FIG. 9B is a schematic block diagram of a plurality of optical transmitters of a single optical transmitter array of FIG. 9A;

FIG. 9C is a is a plot of an estimated blood absorption of an optical signal versus wavelength of the optical signal output by a single optical transmitter of FIG. 9B;

FIG. 9D is a table showing exemplary wavelengths, carrier frequencies and signal paths of optical signals corresponding to certain optical transmitters and optical receivers depicted in FIG. 9A; and

FIG. 10 is a flow chart of at least a portion of the steps of a process for filtering and demodulating a broadband electronic signal received from the user's skin to generate PPG electronic signals in accordance with embodiments of the technology.

The drawing figures do not limit the present technology to the specific embodiments disclosed and described herein. The drawings are not necessarily to scale, emphasis instead being placed upon clearly illustrating the principles of the technology.

### DETAILED DESCRIPTION OF THE TECHNOLOGY

The following detailed description of the technology references the accompanying drawings that illustrate specific embodiments in which the technology can be practiced. The embodiments are intended to describe aspects of the technology in sufficient detail to enable those skilled in the art to practice the technology. Other embodiments can be utilized and changes can be made without departing from the scope of the present technology. The following detailed description is, therefore, not to be taken in a limiting sense. The scope of the present technology is defined only by the appended claims, along with the full scope of equivalents to which such claims are entitled. Furthermore, the word “voltage” may be used to describe electric voltage and the word “current” may be used to describe electric current.

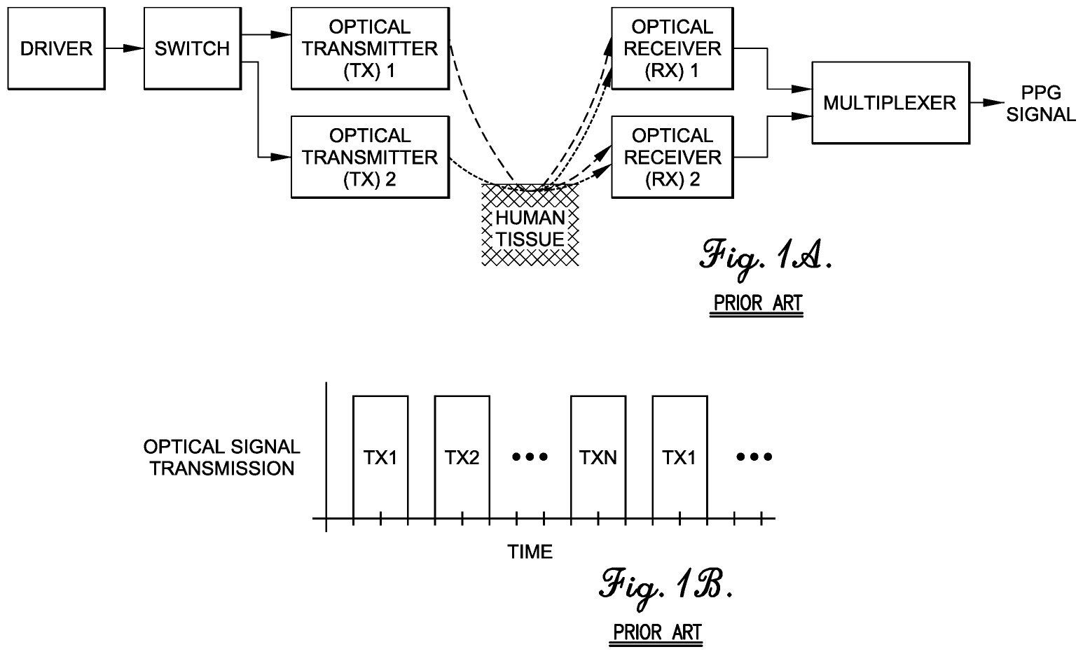

Referring to FIG. 1A, conventional wrist-worn electronic wearable devices typically include circuitry and components, such as a driver, a switch, at least a first optical transmitter (TX) and a second optical transmitter, at least a first optical receiver (RX) and a second optical receiver, and a multiplexer, to generate optical signals that are output into a user's skin, received by a plurality of optical receivers and used to generate a PPG electronic signal that can be used to determine physiological information about the user. The physiological information may include measuring a user's pulse or heart rate, a pulse oximetry (“Pulse Ox”) level (also known as a level of blood oxygen saturation, or SpO2), an estimated stress level, a maximum rate of oxygen consumption (VO2 max), or the like. Typically, a signal processor of the conventional wrist-worn electronic wearable device controls operation of each of a plurality of optical transmitters and stores in a memory element of the wrist-worn electronic wearable device information such as a time window and a power level of optical signals output by each optical transmitter that is subsequently used by the signal processor to determine and identify one of the plurality of optical transmitters that was configured to transmit its optical signal during a given time period in which an optical receiver received an optical signal from the user's skin. The signal processor of the conventional wrist-worn electronic wearable device may utilize the determined source of an optical signal to identify a signal path through which the optical signal passed from one of the plurality of optical transmitters to the optical receiver. This conventional time division multiplexing (TDM) technique for utilizing a plurality of optical transmitters enables the signal processor of the of the conventional wrist-worn electronic wearable device to control the plurality of optical transmitters to select a location of an optical transmitter and a wavelength of the optical signal that is desired to be output into the user's skin at a specified time. As the number of sequential optical transmissions increase, the duration of time required for all transmissions to occur increases, which extends the minimum period of time between successive optical signal transmissions in the sequence. Additionally, such conventional TDM techniques limit the number of optical signals that may be output by the plurality of optical transmitters to a sequence in which only one optical signal output by one of the plurality of optical transmitters travels along a signal path to an optical receiver in an area of the user's skin for each time period. Accordingly, a separate time window exists for each transmission and, in some cases, simultaneous optical transmissions require separating the areas of the user's wrist through which optical signals that are output simultaneously may pass through (along signal paths between optical transmitters and optical receivers) for each time period in order for the signal processor of the conventional wrist-worn electronic wearable device to determine and identify one of the plurality of optical transmitters that was configured to transmit its optical signal proximate to the optical receiver that received an optical signal from the user's skin during the time period in which the optical signal was received.

The driver of the conventional wrist-worn electronic wearable device typically outputs a driver electronic signal, which is commonly a fixed, constant current level during a sampling period. The switch typically electrically couples the driver with the plurality of optical transmitters positioned on a bottom wall of the wrist-worn electronic wearable device. In some cases, the switch receives the driver electronic signal and outputs the driver electronic signal to the first optical transmitter or the second optical transmitter according to control settings that select use of its first output port, which directs the driver electronic signal to the first optical transmitter, or the second output port, which directs the driver electronic signal to the second optical transmitter. Once the first optical transmitter receives the driver electronic signal, it outputs (emits) a first optical signal into the user's wrist. Similarly, once the second optical transmitter receives the driver electronic signal, it outputs (emits) a second optical signal into the user's wrist. Each optical signal is formed from electromagnetic radiation having a wavelength in the violet, visible light, and/or infrared spectrums and has an optical intensity level corresponding to the current level of the driver electronic signal, which corresponds to the driver electronic signal. The optical signals pass through, and/or are reflect from, the user's skin (human tissue), where they are modified, influenced or otherwise impacted, by the amount or flow of blood, or other characteristics of blood vessels, in the signal path of the optical signal. Specifically, the optical signal is modified influenced or otherwise impacted by the blood flow in response to the beating of the user's heart, or the cardiac cycle. After interacting with the user's skin, the optical signals are received by the first optical receiver and the second optical receiver of the conventional wrist-worn electronic wearable device. The first optical receiver typically outputs an analog first PPG electronic signal corresponding to the intensity of the optical signal received, and the second optical receiver typically outputs an analog second PPG electronic signal corresponding to the intensity of the optical signal received. The multiplexer of the conventional wrist-worn electronic wearable device receives each of the PPG electronic signals and outputs a selected one of the PPG electronic signals according to control settings. In some cases, the conventional wrist-worn electronic wearable device includes an analog to digital converter to convert one or more of the analog electronic signals of the device to digital data values, or streams of digital data values.

In general, multiple optical transmitters may be utilized for a couple of reasons. A first reason may be to position multiple optical transmitters at various locations relative to one or more optical receivers in order to cause the optical signals to pass along certain desired signal paths through the user's skin to one or more optical receivers. As the PPG electronic signals that pass through different optical signal paths may have different characteristics, when analyzed in combination, the diversity of signal paths may increase the accuracy of physiological information determined using the PPG electronic signals. A second reason may be to provide additional information about the user's cardiac parameters. For example, optical signals of different wavelengths may be utilized to determine the user's pulse oximetry (“Pulse Ox”) level (blood oxygen saturation, or SpO2), a maximum rate of oxygen consumption (VO2 max), and the like. A signal processor receiving and using the PPG electronic signals to determine physiological information for the user may need to identify the source (optical transmitter) of each PPG electronic signal to determine the wavelength of the optical signal, the signal path along which the optical signal passed and when the optical signal was output by the optical transmitter in order to perform the analysis necessary to determine the physiological information for the user.

Accordingly, prior art electronic wearable devices utilize a time division multiplexing (TDM) optical signal transmission technique in which each optical transmitter outputs its optical signal during one of a plurality of predetermined time windows. Referring to FIG. 1B, a processor may control a first optical transmitter (TX1) to output its optical signal during a first time window, a second optical transmitter (TX2) to output its optical signal during a second time window, and so forth, until each optical transmitter (TXN) has output its optical signal, at which point, the process repeats. Each time window is typically a short period of time, such as less than 1 millisecond or 5 milliseconds. The processor of the of the conventional wrist-worn electronic wearable device typically controls each optical transmitter to generate the optical signal as a desired a fixed or constant (DC) level of intensity (optical signals of different magnitude may be output by the plurality of optical transmitters).

The TDM optical signal transmission approach creates a dilemma. It is desirable to leave each optical transmitter on, transmitting the optical signal, for as long as possible to achieve the highest signal quality and to allow time for the processor to process the information from the PPG electronic signal. However, the more time that any one optical transmitter is on results in the more time that all of the other optical transmitters are off. Thus, there is a conflicting desire to completing more cycles of optical signal transmissions to improve accuracy of physiological information determined using the PPG electronic signals and outputting each optical signal for an extended duration, which would extend the duration of time required for all of the optical transmitters to output an optical signal during their respective time window, in order to collect longer duration, continuous data in each cycle of optical signal transmissions. Similarly, there is a conflicting desire to collecting longer duration, continuous data in each cycle of optical signal transmissions and outputting signals for a shorter duration, which would reduce the duration of time required for all of the optical transmitters to output an optical signal during their respective time window, in order to complete more cycles of optical signal transmissions. These conflicts ultimately limit the number of optical transmitters that a processor can control to output optical signals to a single optical receiver and the duration of each optical signal transmission. For example, referring to FIG. 1B, if “N” is a fifth optical transmitter and each of the five time windows are 1 millisecond in duration, a complete sequence of transmissions would occur in 5 milliseconds and three sequences of transmissions would be completed in 15 milliseconds. The accuracy of physiological information determined using the PPG electronic signals would be improved if each of the five optical transmitters could simultaneously output optical signals into a common area of the user's skin either continuously (in comparison to the example above, resulting in all five optical transmitters simultaneously transmitting optical signals in 1 millisecond) or for a predetermined duration of time that limits power consumption.

Embodiments of the present invention utilize modulation of optical signals at different (non-overlapping), predetermined frequencies, signal phases and/or waveforms to enable the multiplexing of the larger number of optical signals. Specifically, a processor of an electronic wearable device may control a plurality of modulators to implement a modulation function that results in the output of a modulated electronic signal having a predetermined frequency, phase and/or waveform that enables each optical signal, and each optical transmitter from which the optical signal was output, to be distinguished from optical signals output by other optical transmitters. For each optical signal received by an optical receiver, the processor may determine the optical transmitter from which the optical signal was output, the signal path along which the optical signal passed from the optical transmitter to the optical receiver and physiological information about the user based on the optical signal and the determined signal path along which the optical signal travelled through the user's skin.

For example, in embodiments, the improvements disclosed herein enable a processor to control modulators to generate carrier signals having different frequencies and a plurality of optical transmitters (e.g., LEDs) to enable all of the optical transmitters to output optical signals over a plurality of signal paths. The duration for which each optical transmitter outputs an optical signal may be continuous or limited to a period of time that satisfies the applicable signal-to-noise requirements to accurately determine cardiac information about the user. For instance, modulators or electrical circuitry coupled with the plurality of optical transmitters may modulate a drive current input to each optical transmitter in order for the output optical signals to include a carrier frequency, which may be different than a carrier frequency associated with other optical transmitters. As the optical signal output by each optical transmitter passes through the tissue on an upper surface of the user's skin, such as the upper skin of the user's wrist, the blood attenuates the optical signal (typically, the amplitude of the modulated optical signal reflects a volume or concentration of blood present in a region and/or layer of the user's skin tissue) and an optical receiver (e.g., photodiode) outputs a signal that, once demodulated, results in the generation of a PPG electronic signal for the user that corresponds to the amplitude of the received optical signal (at a given carrier frequency) and is used by a signal processor to determine physiological information about the user. An optical receiver that receives multiple optical signals each having a predetermined carrier frequency outputs a broadband optical signal that includes a plurality of optical signals that are each associated with a carrier frequency. The use of a unique carrier frequency that corresponds to a particular optical transmitter by location and wavelength enables a signal processor to evaluate and process each demodulated PPG electronic signal that is output by an optical receiver and determine and select the optical transmitter from which each signal originated. In embodiments, the signal processor may determine and utilize the signal path along which the optical signal associated with a PPG electronic signal passed (from the optical transmitter to the optical receiver) to select the PPG electronic signal for use with determining the physiological information about the user.

Accordingly, in embodiments, the processor may be configured to control a plurality of optical transmitters to each output an optical signal simultaneously either continuously or for a predetermined duration of time, receive one or more broadband electronic signals from one or more optical receivers, filter and demodulate the broadband electronic signals to generate PPG electronic signals, utilize a stored predetermined carrier frequency to determine the optical transmitter from which each optical signal corresponding to a PPG electronic signal was output and a signal path along which the optical signal traveled to the optical receiver that received the broadband electronic signal containing the optical signal associated with a predetermined carrier frequency. In embodiments, the processor may be configured to select one or more signal paths and associated PPG electronic signals for use with determining physiological information about the user. The processor being configured to control each optical transmitter to utilize a predetermined carrier frequency enables frequency division multiplexing of optical signals transmitted by a plurality of optical transmitters, which allows the processor to control one or more (or all) of the optical transmitters to output an optical signal simultaneously and continuously. Such enhancements enable improved accuracy in determining physiological information such as a user's heart rate, pulse oximetry and other parameters.

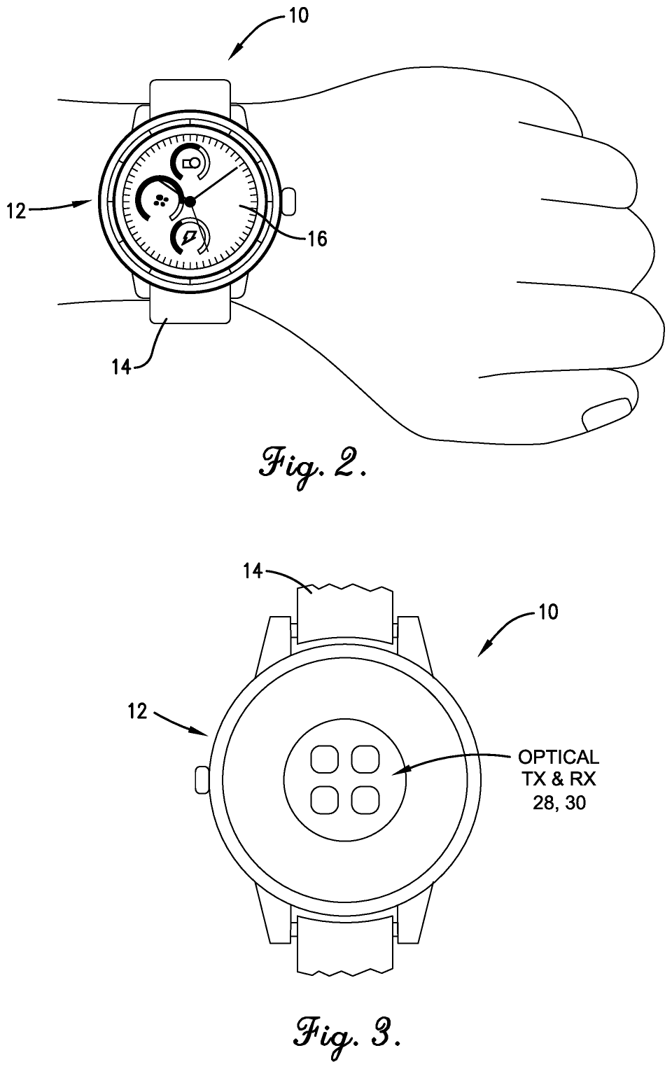

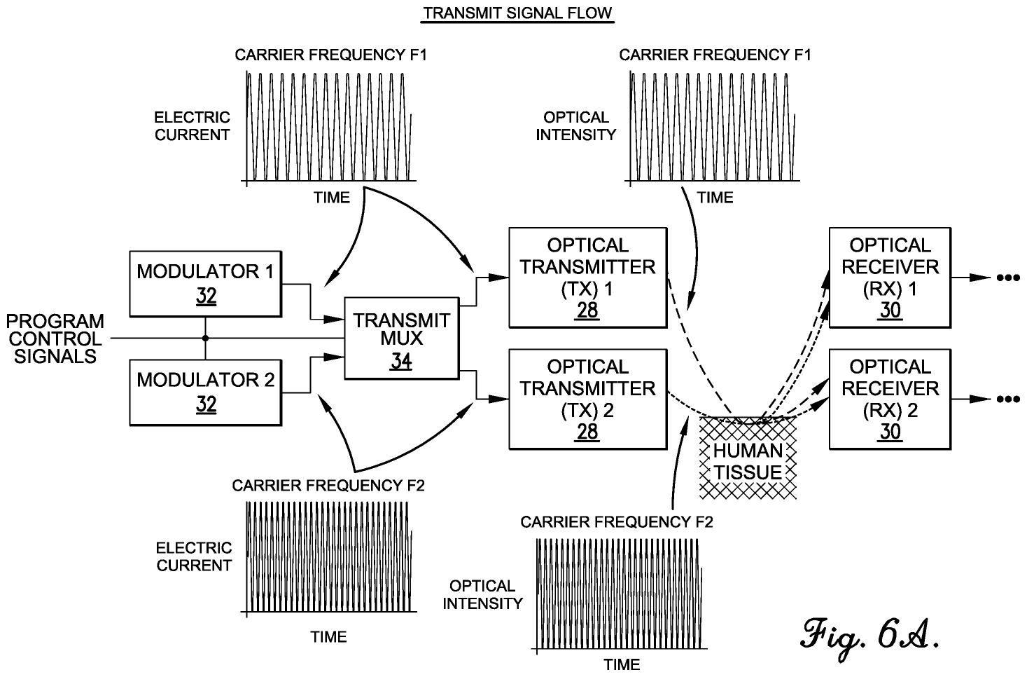

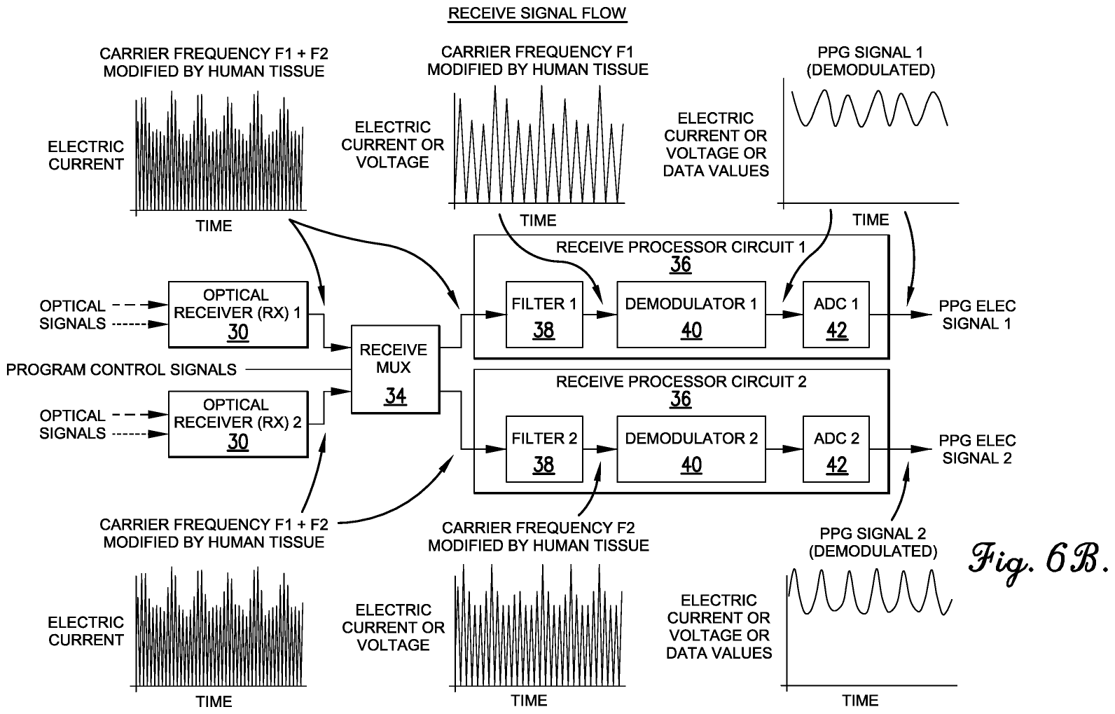

Referring to FIGS. 2-8, embodiments of the present technology provide an electronic wearable device, typically worn on a user's wrist or otherwise secured against the user's skin on a different portion of the user's body, that enables a plurality of optical transmitters to simultaneously output optical signals into a common area of the user's skin either continuously or for a predetermined duration of time. The electronic wearable device includes a plurality of optical transmitters, each of which emits an optical signal having wavelength and a periodic waveform that has a predetermined (selectively unique) carrier frequency. That is, a first optical transmitter emits a first optical signal having a first carrier frequency, a second optical transmitter emits a second optical signal having a second carrier frequency which is different from the first carrier frequency, and so forth for each optical transmitter. This configuration allows for the source optical transmitter for each optical signal to be uniquely identified by its carrier frequency, which in turn, allows for all optical signals to be simultaneously transmitted continuously or as desired and a signal path from the optical transmitter to an optical receiver to be taken into account when determining physiological information about the user.

The electronic wearable device also includes a plurality of optical receivers, each of which receives optical signals that passed through and/or reflected by the user's skin as a broadband electronic signal that includes the optical signals. It is to be understood that each optical receiver receives a broadband electronic signal containing optical signals associated with different carrier frequencies and the processor is configured to receive from a receive-side multiplexer a plurality of analog broadband electronic signals (each received from an optical receiver and output by the optical receiver) and convert (digitize) each analog broadband electronic signal into a digital broadband electronic signal using an analog to digital converter (ADC).

The electronic wearable device may further include a plurality of receive processor circuits—typically, one receive processor circuit for each optical signal that is received by the optical receiver. Each receive processor circuit receives the broadband electronic signal, filters out signals other than an optical signal having a carrier frequency, demodulates the filtered signal in order to extract the amplitude of the carrier frequency, and outputs the PPG electronic signal, which includes the characteristics imparted on the optical signal by traveling through the user's skin.

Alternatively, the electronic wearable device may further include an analog to digital converter configured to receive the analog broadband electronic signal output by the optical receiver and output the broadband electronic signal as a stream of digital data values corresponding to the broadband electronic signal and a processor that filters the broadband electronic signal and demodulates the filtered optical signal to output the PPG electronic signal, which includes the characteristics imparted on the optical signal by traveling through the user's skin.

The components of the electronic wearable device provide a frequency division multiplexing (FDM) optical signal transmission in which the optical transmitters can operate simultaneously and continuously, or for a predetermined duration of time that limits power consumption, allowing for increased accuracy in a processor using the PPG electronic signals to determine physiological information for the user. The processor is configured to determine physiological information about the user based on one or more selected PPG electronic signals and the signal path along which an optical signal associated with each selected PPG electronic signal traveled from an optical transmitter to an optical receiver. For example, the processor may be configured to determine physiological information about the user based on PPG electronic signals associated with optical signals that traveled along substantially perpendicular signal paths. In such an example, the processor may select optical transmitters and optical receivers between which optical signals would travel along a substantially perpendicular path, determine a predetermined carrier frequencies associated with the selected optical transmitters, select PPG electronic signals that correspond to the predetermined carrier frequencies and determine physiological information about the user based on the selected PPG electronic signals. In embodiments, the electronic wearable device may include thirty-two optical transmitters and the processor may be configured to selectively control each optical transmitter to output optical signals having one of thirty-two predetermined carrier frequencies (or signal phases and/or waveforms) to enable the multiplexing of optical signals output by the thirty-two optical transmitters.

Embodiments of the technology will now be described in more detail with reference to the drawing figures. An exemplary electronic wearable device 10 may be embodied by a smart watch or a wearable band that is typically worn on a user's wrist, but may also be embodied by bands or belts worn on the user's arm, leg, torso, or skin of other portions of the user's body. Other examples of the electronic wearable device 10 may include smartphones, personal data assistants, or the like which include a surface, operable to retain optical devices, that can be pressed against the user's skin. Referring to FIGS. 2-5, the electronic wearable device 10 broadly comprises a housing 12, a wrist band 14, a display 16, a user interface 18, a communication element 20, a location determining element 22, a memory element 24, and a processor 26. The electronic wearable device 10 further comprises a plurality of optical transmitters 28 and a plurality of optical receivers 30 on a bottom wall of the electronic wearable device 10. In embodiments, the electronic wearable device 10 includes optical signal processing circuitry.

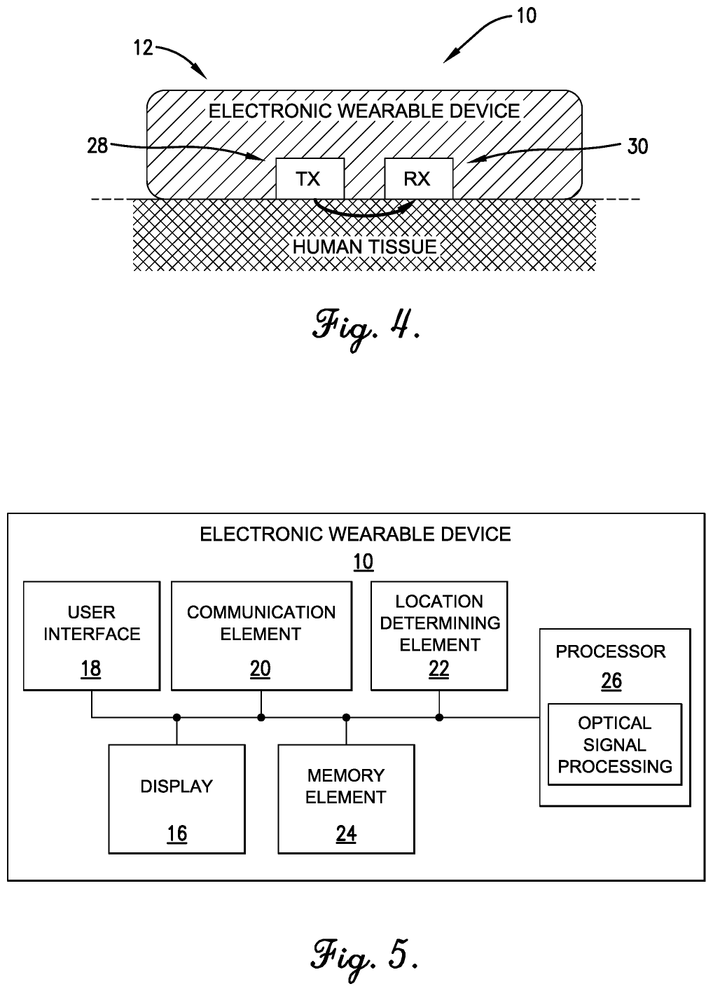

The housing 12, as shown in FIGS. 2, 3, and 4, generally houses or retains other components of the electronic wearable device 10 and may include or be coupled to the wrist band 14. The housing 12 may include a bottom wall, an upper surface, and at least one side wall that bound an internal cavity (not shown in the figures). The bottom wall includes a lower, outer surface that contacts the user's skin, such as the upper layers of the user's wrist, while the user is wearing the electronic wearable device 10. The upper surface opposes the bottom wall. In various embodiments, the upper surface may further include an opening that extends from the upper surface to the internal cavity. In some embodiments, such as the exemplary embodiments shown in the figures, the bottom wall of the housing 12 may have a round, circular, or oval shape, with a single circumferential side wall. In other embodiments, the bottom wall may have a four-sided shape, such as a square or rectangle, or other polygonal shape, with the housing 12 including four or more sidewalls. Referring to FIGS. 3 and 4, the bottom wall may include one or more openings in which the plurality of optical transmitters 28 and the plurality of optical receiver 30 are placed, positioned, or located. The one or more openings within the bottom wall may be covered by one or more lenses (not shown in the figures) through which the optical signal may be transmitted and received.

The display 16, as shown in FIG. 2, generally presents the information mentioned above, such as time of day, current location, and the like. The display 16 may be implemented in one of the following technologies: light-emitting diode (LED), organic LED (OLED), Light Emitting Polymer (LEP) or Polymer LED (PLED), liquid crystal display (LCD), thin film transistor (TFT) LCD, LED side-lit or back-lit LCD, or the like, or combinations thereof. In some embodiments, the display 16 may have a round, circular, or oval shape. In other embodiments, the display 16 may possess a square or a rectangular aspect ratio which may be viewed in either a landscape or a portrait orientation.

The user interface 18 generally allows the user to directly interact with the electronic wearable device 10 and may include pushbuttons, rotating knobs, crowns, or the like. In various embodiments, the display 16 may also include a touch screen occupying the entire display 16 or a portion thereof so that the display 16 functions as at least a portion of the user interface 18. The touch screen may allow the user to interact with the electronic wearable device 10 by physically touching, swiping, or gesturing on areas of the display 16.

The communication element 20 generally allows communication with external systems or devices. The communication element 20 may include signal and/or data transmitting and receiving circuits, such as antennas, amplifiers, filters, mixers, oscillators, digital signal processors (DSPs), and the like. The communication element 20 may establish communication wirelessly by utilizing radio frequency (RF) signals and/or data that comply with communication standards such as cellular 2G, 3G, 4G, LTE, or 5G, Institute of Electrical and Electronics Engineers (IEEE) 802.11 standard such as Wi-Fi, IEEE 802.16 standard such as WiMAX, Bluetooth™, or combinations thereof. In addition, the communication element 20 may utilize communication standards such as ANT, ANT+, Bluetooth™ low energy (BLE), the industrial, scientific, and medical (ISM) band at 2.4 gigahertz (GHz), or the like. Alternatively, or in addition, the communication element 20 may establish communication through connectors or couplers that receive metal conductor wires or cables which are compatible with networking technologies such as Ethernet. In certain embodiments, the communication element 20 may also couple with optical fiber cables. The communication element 20 may be in electronic communication with the memory element 24 and the processor 26.

The location determining element 22 generally determines a current geolocation of the electronic wearable device 10 and may receive and process radio frequency (RF) signals from a multi-constellation global navigation satellite system (GNSS) such as the global positioning system (GPS) utilized in the United States, the Galileo system utilized in Europe, the GLONASS system utilized in Russia, or the like. The location determining element 22 may accompany or include an antenna to assist in receiving the satellite signals. The antenna may be a patch antenna, a linear antenna, a loop antenna, a slot antenna, or any other type of antenna that can be used with location or navigation devices. The location determining element 22 may include satellite navigation receivers, processors, controllers, other computing devices, or combinations thereof, and memory. The location determining element 22 may process a location electronic signal communicated from the antenna which receives the location wireless signal from one or more satellites of the GNSS. The location wireless signal includes data from which geographic information such as the current geolocation is derived. The current geolocation may include coordinates, such as the latitude and longitude, of the current location of the electronic wearable device 10. The location determining element 22 may communicate the current geolocation to the processor 26, the memory element 24, or both.

Although embodiments of the location determining element 22 may include a satellite navigation receiver, it will be appreciated that other location-determining technology may be used. For example, cellular towers or any customized transmitting radio frequency towers can be used instead of satellites may be used to determine the location of the electronic wearable device 10 by receiving data from at least three transmitting locations and then performing basic triangulation calculations to determine the relative position of the device with respect to the transmitting locations. With such a configuration, any standard geometric triangulation algorithm can be used to determine the location of the electronic wearable device 10. The location determining element 22 may also include or be coupled with a pedometer, accelerometer, compass, or other dead-reckoning components which allow it to determine the location of the electronic wearable device 10. The location determining element 22 may determine the current geographic location through a communications network, such as by using Assisted GPS (A-GPS), or from another electronic wearable device. The location determining element 22 may even receive location data directly from a user.

The memory element 24 may be embodied by devices or components that store data in general, and digital or binary data in particular, and may include exemplary electronic hardware data storage devices or components such as read-only memory (ROM), programmable ROM, erasable programmable ROM, random-access memory (RAM) such as static RAM (SRAM) or dynamic RAM (DRAM), cache memory, hard disks, optical disks, flash memory, thumb drives, universal serial bus (USB) drives, solid state memory, or the like, or combinations thereof. In some embodiments, the memory element 24 may be embedded in, or packaged in the same package as, the processor 26. The memory element 24 may include, constitute, or embody, a non-transitory “computer-readable medium”. The memory element 24 may store the instructions, code, code statements, code segments, software, firmware, programs, applications, apps, services, daemons, or the like that are executed by the processor 26. The memory element 24 is in electronic communication with the processor 26 and may also store the optical signals output by each optical receiver, a carrier frequency corresponding to each optical transmitter (and optical signal output by the optical transmitter) and data that is received by the processor 26 or the device in which the processor 26 is implemented. The processor 26 may further store data or intermediate results generated during processing, calculations, and/or computations as well as data or final results after processing, calculations, and/or computations. In addition, the memory element 24 may store settings, databases, and the like.

The processor 26 may comprise one or more processors. The processor 26 may include electronic hardware components such as microprocessors (single-core or multi-core), microcontrollers, digital signal processors (DSPs), field-programmable gate arrays (FPGAs), analog and/or digital application-specific integrated circuits (ASICs), intelligence circuitry, or the like, or combinations thereof. The processor 26 may generally execute, process, or run instructions, code, code segments, code statements, software, firmware, programs, applications, apps, processes, services, daemons, or the like. The processor 26 may also include hardware components such as registers, finite-state machines, sequential and combinational logic, configurable logic blocks, and other electronic circuits that can perform the functions necessary for the operation of the present technology. In certain embodiments, the processor 26 may include multiple computational components and functional blocks that are packaged separately but function as a single unit. In some embodiments, the processor 26 may further include multiprocessor architectures, parallel processor architectures, processor clusters, and the like, which provide high performance computing. The processor 26 may be in electronic communication with the other electronic components of the electronic wearable device 10 through serial or parallel links that include universal busses, address busses, data busses, control lines, and the like. In addition, the processor 26 may include analog to digital converters (ADCs) to convert analog electronic signals to digital data values, or streams of digital data values, and/or digital to analog converters (DACs) to convert digital data values, or streams of digital data values, to analog electronic signals.

Referring to FIG. 6A and 6B, a transmit signal flow and a receive signal flow are illustrated. In addition to the plurality of optical transmitters 28 and the plurality of optical receiver 30, the electronic wearable device 10 further comprises a plurality of modulators 32, a plurality of multiplexers 34 (MUX), and a plurality of receive processor circuits 36. The transmit signal flow includes a plurality of modulators 32 and a plurality of optical transmitters 28 outputting optical signals into the user's skin that are received by at least one of a plurality of optical receivers 30. The receive signal flow includes a plurality of receive processor circuits 36 filtering and demodulating optical signals that one or more optical receivers 30 received from the user's skin to generate PPG electronic signals. It is to be understood that the processor 26 may execute, process, or run instructions to perform the functions described herein for the receive processor circuits 36, such as converting analog optical signals output by the plurality of optical receivers 30 to digital data values, or streams of digital data values the optical signals using internal or external analog to digital converters (ADCs).

Each modulator 32 includes electric circuitry such as signal generators, signal amplifiers, signal filters, and so forth, that are configured to output a modulated electronic signal that has a periodic waveform having a carrier frequency. For example, the modulator 32 may include a signal generator that outputs a periodically varying electric voltage and a transconductance amplifier that receives the voltage and converts it to a periodically varying electric current. The amplitude of the varying electric current is selected according to electrical characteristics of each optical transmitter 28. The frequency value of the carrier frequency is selectively variable and out of band with the human biological signals, which are typically about 4 Hertz (Hz) or less. Thus, the value of the carrier frequency may range from approximately 1 kiloHertz (kHz) to approximately 10 kHz or greater. The waveform may have a shape selected from a plurality of shapes including sinusoidal, square, triangular, and the like. Each modulator 32 is controlled by processor 26, which may transmit a program control signal to each modulator 32, to select a carrier frequency of the modulated electronic signal. The processor 32 may store the carrier frequency selected for each modulator 32, which may correspond to one or more optical transmitters 28, in the memory element 24. The program control signal output by the processor 32 may also select the type of waveform for the modulated electronic signal. In some embodiments, the electronic wearable device 10 may include a modulator 32 for each optical transmitter 28. In other embodiments, the electronic wearable device 10 may include circuitry to multiply or otherwise modify a modulated electronic signal to generate a plurality of modulated electronic signals for the plurality of optical transmitters 28. The processor 26 may also control the timing for when each modulator 32 will output the modulated electronic signal and other parameters of each modulator 32.

Each multiplexer 34 includes electric circuitry such as active and/or passive switching elements capable of being configured or programmed to receive an electronic signal and pass it to one of a plurality of outputs. Each multiplexer 34 includes a plurality of input ports and a plurality of output ports, wherein the signal of each input can be routed to any one of the output ports. The processor 26 outputs to each multiplexer 34 program control signals that select the output port for each input signal. In embodiments, the electronic wearable device 10 includes a first multiplexer 34 to operate in the transmit signal flow and a second multiplexer 34 to operate in the receive signal flow. The first multiplexer 34 is configured to electrically couple an input port for each modulator 32 and an output port for each optical transmitter 28. Similarly, the second multiplexer 34 is configured to electrically couple an input port for each optical receiver 30 and an output port for each receive processor circuit 36. In embodiments, the second multiplexer 34 that receives broadband electronic signals from an optical receiver 30 may output the electronic signals to a transimpedance amplifier that receives a periodically varying electric current and converts it to a voltage.

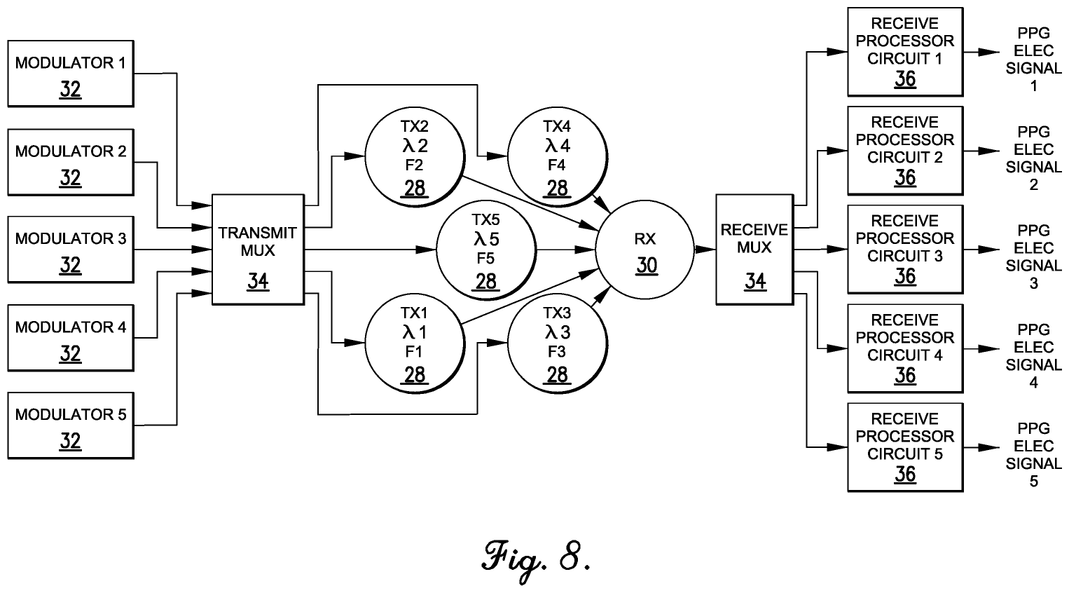

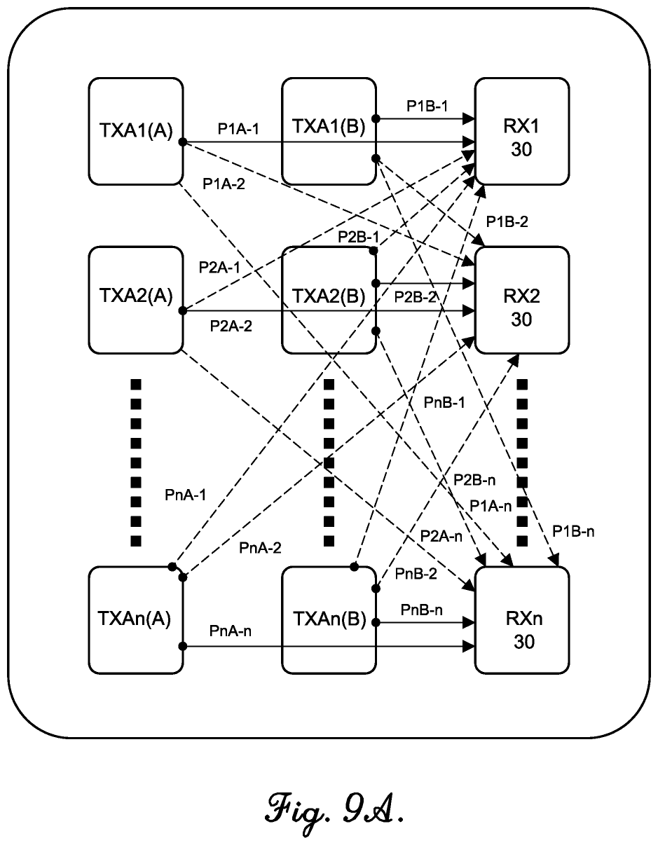

Each optical transmitter 28 includes an electronic device such as a light emitting diode (LED), or other short wavelength EM radiation emitting device, that is configured to receive an electronic signal (typically of varying electric current) and transmit, or output (emit), an optical signal including light, i.e., EM radiation corresponding in amplitude and frequency to the electronic signal and having a wavelength (λ) in the ultraviolet, visible light, or infrared spectrum. The photonic generator of each optical transmitter transmits or outputs electromagnetic radiation having a particular wavelength (the optical signal) in the visible light spectrum, which is typically between approximately 400 nanometers (nm) to 700 nm, in the near infrared spectrum, which is typically between approximately 700 nm to 1,000 nm, or in a wavelength range of 1,000 nm to 1,500 nm. The wavelength of the optical signal is generally determined by, or varies according to, the material from which the photonic generator of each optical transmitter is formed. For instance, optical signals having a wavelength between 500 nm and 600 nm are associated with visually appearing as the color green. In some embodiments, some of the optical transmitters 28 may output optical signals having the same wavelength. In other embodiments, each of the plurality of optical transmitters 28 may output optical signals having a different wavelength, as shown in FIG. 8.

Each optical receiver 30 includes an electronic device such as a photodiode, a photo resistor, a phototransistor, or the like which is configured to output, or change, an electrical characteristic according to the intensity of the EM radiation, i.e., the optical signal, impinging the device. For example, each optical receiver 30 may receive transmissions or reflections of the optical signal from the skin and output an electric current that corresponds to (varies according to) the intensity of the received optical signal. Specifically, the optical receiver 30 outputs a broadband electronic signal having an amplitude and a frequency that varies according to the amplitude and frequency, i.e., the carrier frequency, of the received optical signal. In embodiments of the electronic wearable device 10, the optical receiver 30 receives optical signals from all of optical transmitters 28 within proximate to the optical receiver 30 such that each optical signal travels along a distinct signal path from an optical transmitter 28 to the optical receiver 30. In such cases, the optical receiver 30 acts like a signal mixer such that the amplitude and frequency of the broadband electronic signal vary according to the component optical signals (as including the frequencies of all of the optical signals received by the optical receiver 30). In addition, the optical receiver 30 is sensitive to the EM radiation from a range of wavelengths including wavelengths in the ultraviolet, visible light, and infrared spectrums.

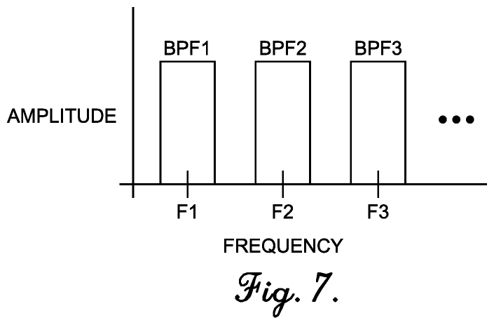

In embodiments, each PPG processor circuit 36 receives the broadband electronic signal from an optical receiver 30 or a receive multiplexer 34 and outputs a PPG electronic signal. The PPG processor circuit 36 may include a filter 38, a demodulator 40, and an ADC 42. The filter 38 includes electric circuitry such as a band pass filter (BPF) that is configured to pass frequency components of the broadband electronic signal that have a frequency value substantially equal to the carrier frequency. Accordingly, the filter 38 filters out any frequencies other than the carrier frequency. For example, referring to FIG. 7, a first filter 38 (BPF1) passes a first band of frequencies centered at carrier frequency F1 and filters out frequency components that lie outside the first band, a second filter 38 (BPF2) passes a second band of frequencies centered at carrier frequency F2 and filters out frequency components that lie outside the second band, a third filter 38 (BPF3) passes a third band of frequencies centered at carrier frequency F3 and filters out frequency components that lie outside the third band, and so forth. Each filter 38 receives the broadband electronic signal and outputs a filtered electronic signal that includes the carrier frequency component associated with the frequency selected by the band pass filter frequency. The filtered electronic signal is the optical signal that has been modified influenced or otherwise impacted by traveling through human tissue and interacting with blood vessels, having an applicable carrier frequency. That is, the filter 38 of the first PPG processor circuit 36 outputs a first filtered electronic signal that includes the first carrier frequency component modified by human tissue, the filter 38 of the second PPG processor circuit 36 outputs a second filtered electronic signal that includes the second carrier frequency component modified by human tissue, the filter 38 of the third PPG processor circuit 36 outputs a third filtered electronic signal that includes the third carrier frequency component modified by human tissue, and so forth.

The demodulator 40 includes electric circuitry such as an envelope detector including rectification and filtering or the like that is configured to extract the amplitude of the carrier frequency component from the filtered electronic signal—leaving an electronic signal without the carrier frequency while retaining the effects that the human tissue had on the optical signal. Each demodulator 40 receives one of the filtered electronic signals and outputs one of a plurality of PPG electronic signals in analog form that includes the effects that human tissue had on the optical signal traveling along a particular signal path. For example, a first demodulator 40 receives the first filtered electronic signal and outputs a first (analog) PPG electronic signal that includes the effects that human tissue had on the first optical signal traveling along a signal path between the first optical transmitter 28 and the first optical receiver 30. Similarly, a second demodulator 40 receives the second filtered electronic signal and outputs a second PPG electronic signal that includes the effects that human tissue had on the second optical signal traveling along a signal path between the second optical transmitter 28 and the second optical receiver 30.

The ADC 42 includes one or more types of known ADC implementations. The ADC 42 receives the PPG electronic signal, samples it, and outputs the PPG electronic signal as a stream of digital data values that represent the PPG waveform.

In embodiments, processor 26 may execute, process, or run instructions to perform the functions described herein for the receive processor circuits 36. For example, processor 26 may receive the broadband electronic signal from an optical receiver 30 or a receive multiplexer 34 and convert analog electronic signals output by the plurality of optical receivers 30 to digital data values, or streams of digital data values of the electronic signals using an internal ADC or external ADC 42. Once processor 26 generates a digital optical signal, processor 26 may implement a band pass filter (BPF), as a software process and/or configurable logic hardware, that is configured to pass frequency components of the broadband electronic signal that have a frequency value substantially equal to the carrier frequency. Additionally, processor 26 may demodulate the filtered data by removing the carrier frequency component using a software process and/or configurable logic hardware (leaving the filtered data without the carrier frequency component while retaining the effects that the human tissue had on the optical signal). Specifically, processor 26 may receive a filtered electronic signal and output a PPG electronic signal in digital form that includes the effects that human tissue had on the optical signal traveling along a particular signal path.

Referring again to FIGS. 6A and 6B, the electronic wearable device may be configured to operate as follows. Each modulator 32 outputs the modulated electronic signal having a carrier frequency selected by processor 26, which outputs program control signals to each modulator 32 to select the carrier frequency. The modulated electronic signal may have a current with an amplitude that is modulated to correspond to the selected carrier frequency, although the voltage or other electrical characteristics of the modulated electronic signal may also be modulated by modulator 32. In exemplary embodiments, a first modulator 32 outputs a first modulated electronic signal having a first carrier frequency F1, and a second modulator 32 outputs a second modulated electronic signal having a second carrier frequency F2. Although FIG. 6A illustrates only two modulators 32 for the purposes of explanation, it is to be understood that additional modulators 32 may be utilized. In some embodiments, modulators 32 may include circuitry that multiplies or otherwise modifies the modulated electronic signal to generate a plurality of modulated electronic signals for the plurality of optical transmitters 28. All of the modulators 32 may output the modulated electronic signals simultaneously and continuously.

In some embodiments, the transmit multiplexer 34 electrically couples modulators 32 and optical transmitters 28. In such embodiments, the transmit multiplexer 34 receives each of the modulated electronic signals and, based on the program control signals, selectively routes the modulated electronic signal from any one of the input ports to any one of the output ports. In the exemplary embodiment shown in the figures, the first modulated electronic signal, received on the first input port, is routed to the first output port, and the second modulated electronic signal, received on the second input port, is routed to the second output port. In other embodiments, the modulators 32 are electrically coupled with optical transmitters 28 without the transmit multiplexer 34. In such embodiments, an optical transmitter 28 receives a modulated electronic signal output by a modulator 32 from the modulator 32.

Each optical transmitter 28 is positioned on the bottom wall and receives the selected modulated electronic signal from the transmit multiplexer 34 output port to which it is connected or directly from a modulator 32. Each optical transmitter 28 outputs an optical signal having an optical intensity that is modulated corresponding to the carrier frequency of the modulated electronic signal. The optical signal output by each optical transmitter 28 is based on the modulated electronic signal output by a corresponding modulator 32 and has the carrier frequency of the modulated electronic signal.

In exemplary embodiments, the first optical transmitter 28 is connected to the first output port of the transmit multiplexer 34 and thus receives the first modulated electronic signal. Accordingly, the first optical transmitter 28 emits the first optical signal having the first carrier frequency F1 output by first modulator 32. Similarly, the second optical transmitter 28 is connected to the second output port of the transmit multiplexer 34 and thus receives the second modulated electronic signal output by the second modulator 32. Accordingly, the second optical transmitter 28 emits the second optical signal having the second carrier frequency F2. Although only two optical transmitters 28 and two modulators 32 are depicted in FIG. 6A for explanation purposes, it is to be understood that the electronic wearable device 10 may include many optical transmitters 28 individually or within optical transmitter arrays, which include a plurality of optical transmitters 28 that output optical signals from a common location on the bottom wall of the electronic wearable device 10. A transmitter array may form a plurality of optical transmitters 28, some of which output (emit) light at a different wavelength than the light output (emitted) by other optical transmitters 28. For example, in embodiments, the electronic wearable device 10 may include six optical transmitter arrays, each optical transmitter array including four optical transmitters 28, resulting in more than twenty-four optical transmitters 28 that output optical signals from six locations on the on the bottom wall of the electronic wearable device 10. Other combinations of optical transmitters 28 in each optical transmitter array and the number of optical transmitter arrays are contemplated herein.

The first optical signal and the second optical signal output by the first optical transmitter 28 and the second optical transmitter 28, respectively, travel through the user's skin (human tissue) and interact with the blood vessels, which modifies, influences or otherwise impacts the optical signals by the amount or flow of blood, or other characteristics of blood vessels, in the signal path of the optical signal. In general, an optical signal output by an optical transmitter typically consists of its distinct sinusoidal carrier frequency before it enters the user's skin. As that optical signal passes through the user's skin, such as the upper skin of the user's wrist, the presence of red blood cells and other objects in the blood vessels that the optical signal passes through attenuates the amplitude of the carrier signal such that the amplitude of the modulated optical signal decreases as a pulse wave of blood passes by the user's wrist (the pulse wave momentarily passing directly under the wrist-worn device in the signal path of the optical signal).

The optical signals that pass through the user's skin are received by the first optical receiver 30 and the second optical receiver 30, each of which are positioned on the bottom wall of the electronic wearable device 10. Each optical signal output by the optical transmitters 28 retains the carrier frequency associated with the modulated electronic signal output by the corresponding modulator 32 while the optical signal travels through the user's skin from each optical transmitter 28 to each optical receiver 30. In some ways, each optical receiver 30 acts as a signal mixer in that each optical receiver 30 receives a plurality of optical signals each having a predetermined carrier frequency, but outputs just one broadband electronic signal that includes all of the optical signals and their corresponding frequency components. In exemplary embodiments, the first optical receiver 30 and second optical receiver 30 receive the first optical signal and the second optical signal and each outputs a broadband electronic signal corresponds to the first optical signal and the second optical signal as well as the carrier frequency of each (that is the first optical signal remains associated with first carrier frequency F1 and the second optical signal remains associated with second carrier frequency F2). The first optical receiver 30 outputs a first broadband electronic signal and the second optical receiver 30 outputs a second broadband electronic signal. Putting aside the different carrier frequencies associated with the first optical signal and the second optical signal, the first broadband electronic signal and the second broadband electronic signal each have different waveform characteristics because the first optical signal traveled along a different path from each optical transmitter 28 to each optical receiver 30. For example, the first optical signal travels along a first signal path from the first optical transmitter 28 to the first optical receiver 30, the first optical signal travels along a second signal path from the first optical transmitter 28 to the second optical receiver 30, the second optical signal travels along a third signal path from the second optical transmitter 28 to the first optical receiver 30, and the second optical signal travels along a fourth signal path from the second optical transmitter 28 to the second optical receiver 30. Each optical signal is modified, influenced or otherwise impacted, by the amount or flow of blood, or other characteristics of blood vessels, in the signal path of the optical signal as it passes through, and/or are reflect from, the user's skin (human tissue) and each optical signal remains in that form as it passes from the user's skin through each optical receiver 30.

In embodiments, the receive multiplexer 34 electrically couples optical receivers 30 and receive processor circuits 36. In such embodiments, the receive multiplexer 34 receives each of the broadband electronic signals and, based on the program control signals received from processor 26, selectively routes the broadband electronic signal from any one of the input ports to any one of the output ports. In the exemplary embodiment shown in the figures, the first broadband electronic signal, received on the first input port, is routed to the first output port, and the second broadband electronic signal, received on the second input port, is routed to the second output port. In other embodiments, each optical receiver 30 outputs a broadband electronic signal that is received by receive processor circuits 36 and/or processor 26.

In embodiments, the filter 38 of each receive processor circuit 36 receives the selected broadband electronic signal from the receive multiplexer 34 output port to which it is connected and outputs a filtered electronic signal that retains the effects of traveling through the user's skin and the carrier frequency of one of the optical signals. That is, each filter 38 passes portions of the selected broadband electronic signal corresponding to a selected carrier frequency and filters out portions of the selected broadband electronic signal corresponding to the other carrier frequencies. Also, the filtered electronic signal may have a varying voltage or a varying current. In exemplary embodiments, the filter 38 of the first receive processor circuit 36 receives the first broadband electronic signal, passes the first carrier frequency F1, and outputs a first filtered electronic signal having portions of the selected broadband electronic signal corresponding to the first carrier frequency F1. Similarly, the filter 38 of the second receive processor circuit 36 receives the second broadband electronic signal, passes the second carrier frequency F2, and outputs a second filtered electronic signal having portions of the selected broadband electronic signal corresponding to the second carrier frequency F2.

The demodulator 40 of each receive processor circuit 36 receives the filtered electronic signal, extracts the amplitude of the carrier frequency component (leaving an optical signal without the carrier frequency while retaining the effects on the waveform from traveling through the user's skin), and outputs the PPG electronic signal in analog form. The PPG electronic signal may have a varying voltage or a varying current. In exemplary embodiments, the demodulator 40 of the first receive processor circuit 36 receives the first filtered electronic signal, extracts the amplitude of the first carrier frequency F1, and outputs a first PPG electronic signal in analog form. Similarly, the demodulator 40 of the second receive processor circuit 36 receives the second filtered electronic signal, extracts the amplitude of the second carrier frequency F2, and outputs a second PPG electronic signal in analog form. Each PPG electronic signal has different waveform characteristics given that the optical signals (from which the PPG electronic signals are derived) traveled along different paths from each optical transmitter 28 to each optical receiver 30.

The ADC 42 of each receive processor circuit 36 receives the PPG electronic signal in analog form, performs an analog to digital conversion, and outputs the PPG electronic signal in digital form as a stream of digital data values. In exemplary embodiments, the first ADC 42 receives the first PPG electronic signal in analog form and outputs the first PPG electronic signal in digital form. Similarly, the second ADC 42 receives the second PPG electronic signal in analog form and outputs the second PPG electronic signal in digital form. The digital data values of each PPG electronic signal are communicated to the processor 26, which uses the PPG electronic signals to determine physiological information about the user, such as measuring a user's pulse or heart rate, a pulse oximetry level (blood oxygen saturation, or SpO2), an estimated stress level, a maximum rate of oxygen consumption (VO2 max) and other parameters.

In other embodiments, the processor 26 may execute, process, or run instructions, code or software to perform the functions described above for each receive processor circuit 36, such as converting analog optical signals output by the plurality of optical receivers 30 to digital data values, or streams of digital data values the optical signals using internal or external analog to digital converters (ADCs). For example, the electronic wearable device 10 may contain analog to digital converters 42 may be configured to receive the analog broadband electronic signal output by the optical receiver 30 and output the broadband electronic signal as a stream of digital data values corresponding to the broadband electronic signal and processor 26 filters the broadband electronic signal and demodulates the filtered optical signal to output a PPG electronic signal that includes the characteristics imparted on the optical signal by traveling through the user's skin. Similarly, the processor 26 may contain analog to digital converters 42 configured to receive the analog broadband electronic signal output by the optical receiver 30 and output the broadband electronic signal as a stream of digital data values corresponding to the broadband electronic signal.

In embodiments, the processor 26 identify and the memory element 24 may store a location and a wavelength of the optical signal output by each optical transmitter 28 for each PPG signal generated from the broadband electronic signals output by each optical receiver 30 after demodulation of the broadband electronic signals or filtered digital broadband electronic signals. For example, for an embodiment in which six different carrier frequencies are used by optical transmitters 28, the processor 26 identify and the memory element 24 may store one of the six carrier frequencies within the broadband electronic signals or filtered digital broadband electronic signals, separate the subcomponent signals based on the identified carrier frequency, demodulate the PPG signal from carrier signal and the evaluate the PPG signal to determine cardiac information for the user based on the identification of the optical transmitter 28 from which that optical signal originated, the location of that optical transmitter 28, the wavelength of optical signals emitted by the optical transmitter 28 and the signal path along which the optical signal passed from the optical transmitter 28 to the optical receiver 30.

Referring to FIG. 8, in various embodiments, the electronic wearable device 10 includes a plurality of optical transmitters 28 (TX), with each optical transmitter 28 emitting an optical signal having one of a plurality of (optical) wavelengths (λ). Utilizing multiple optical transmitters 28 with each emitting at a different wavelength may enable the processor 26 of the electronic wearable device 10 to perform pulse spectrometry to determine cardiac information including pulse oximetry. In order to distinguish one optical signal from another, the optical transmitters 28 still receive a modulated electronic signal and modulate the optical signal with a predetermined carrier frequency. Thus, the electronic wearable device 10 also includes a modulator 32 for each optical transmitter 28 along with a transmit multiplexer 34 or circuitry to multiply or otherwise modify a modulated electronic signal to generate additional modulated electronic signals for each optical transmitter 28. Each modulator 32 outputs the modulated electronic signal having one of a plurality of carrier frequencies, and the multiplexer 34 routes each modulated electronic signal to an optical transmitter 28 selected by processor 26 using program control signals. In exemplary embodiments, a first optical transmitter 28 outputs (emits) a first optical signal having a first wavelength Al and a first carrier frequency F1, a second optical transmitter 28 outputs a second optical signal having a second wavelength λ2 and a second carrier frequency F2, and so forth with a third optical transmitter 28, a fourth optical transmitter 28, and a fifth optical transmitter 28. The five optical transmitters 28 may output (emit) optical signals at particular wavelengths in order to perform pulse spectrometry. Exemplary wavelengths include approximately 540 nanometers (nm), approximately 630 nm, approximately 660 nm, approximately 700 nm, and approximately 940 nm. The optical transmitters 28 may be formed within an optical transmitter array, which include a plurality of optical transmitters 28 that output optical signals from a common location on the bottom wall of the electronic wearable device 10, or otherwise grouped at a common position on the bottom wall of the electronic wearable device 10. The electronic wearable device 10 may include a plurality of optical transmitter arrays, each containing a plurality of optical transmitters 28 that output (emit) an optical signal having a predetermined carrier frequency and a different wavelength than other optical transmitters 28 in the optical transmitter array. The optical transmitters 28 may be positioned in nearly any formation and in nearly any location relative to the optical receiver(s) 30.

The optical signals output by the optical transmitters 28 pass through and/or reflected by the user's skin are received by at least one optical receiver 30, which outputs a broadband electronic signal that is received by five receive processor circuits 36 via a receive multiplexer 34, as shown in FIG. 8, or the processor 26 for filtering and demodulation via an analog to digital converter 42. Each receive processor circuit 36 or the processor 26 outputs a PPG electronic signal in the same manner as described above.

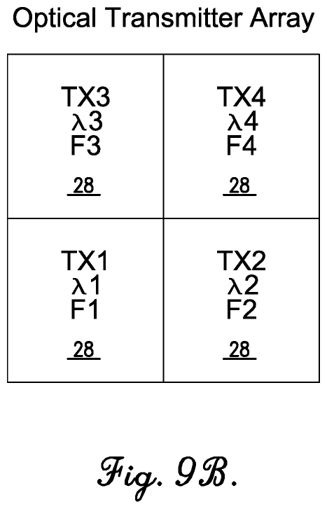

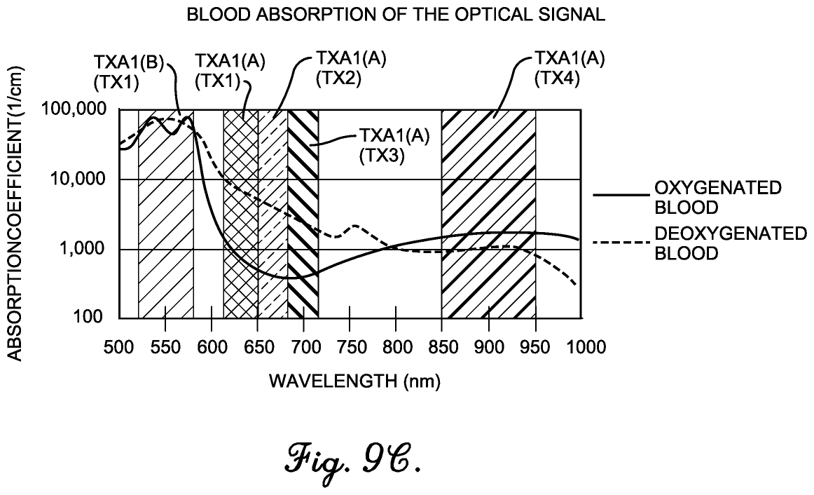

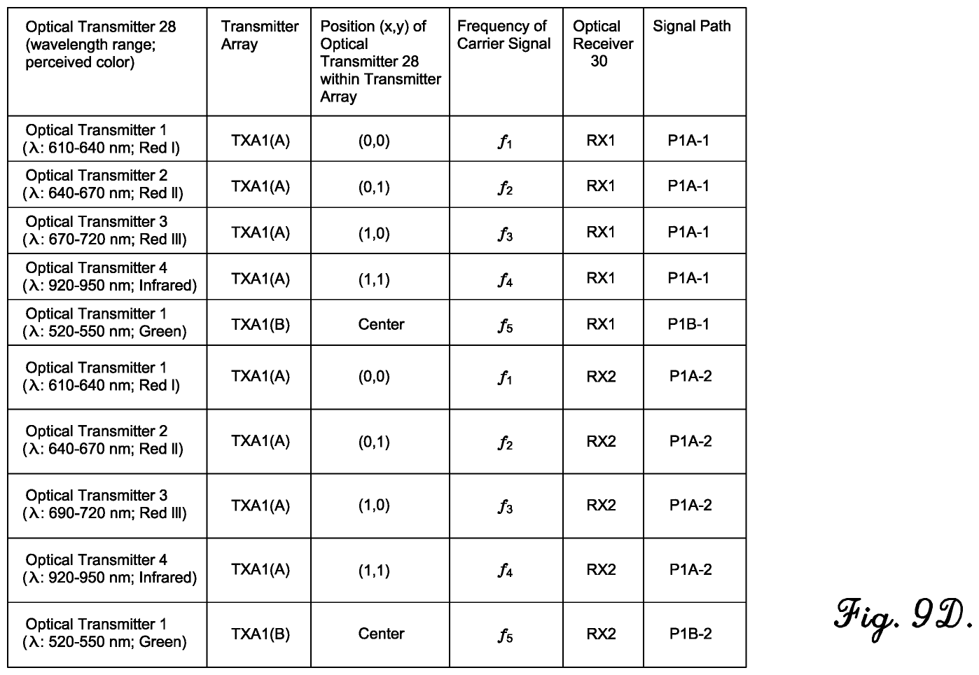

In embodiments, as shown in FIGS. 9A-9F, the electronic wearable device 10 includes a plurality of optical transmitters 28, with each optical transmitter 28 outputting (emitting) an optical signal having one of a plurality of carrier frequencies and one of a plurality of (optical) wavelengths (λ), and a plurality of optical receivers 30. The plurality of optical transmitters 28 and the plurality of optical receivers 30 may be arranged on the bottom wall of the electronic wearable device 10 such that optical signals output by the plurality of optical transmitters 28 may travel to an optical receiver 30 along different signal paths. A plurality of optical transmitters 28 may be positioned at a substantially common position on the bottom wall of the electronic wearable device 10. For instance, an optical transmitter array may include a plurality of optical transmitters 28.

Each optical transmitter array may include a plurality of optical transmitters 28. As shown in FIG. 9B, an optical transmitter array may include four optical transmitters 28 that are positioned relative to each other in a two-dimensional arrangement. The optical signals output by those four optical transmitters 28 will travel along substantially the same signal path to an optical receiver 30. For instance, as shown in FIG. 9A, optical signals output by optical transmitters 28 of a first optical transmitter array TXA1(A) travel along signal path signal path P1A-1 to a first optical receiver (RX1). Similarly, optical signals output by optical transmitters 28 of a second optical transmitter array TXA2(A) travel along signal path signal path P2A-1 to a second optical receiver (RX2).

It is to be understood that many arrangements of optical transmitters 28 and optical receivers 30 are contemplated and that processor 26 may select a carrier frequency for each optical signal output by an optical transmitter 28 that enables the processor 26 to determine the optical transmitter 28 from which each optical signal received by an optical transmitter 30 originated and the signal path along which each optical signal traveled to an optical receiver 30 that output a broadband electronic signal.

In some embodiments, as shown in FIG. 9D, each signal path between an optical transmitter 28 and each optical receiver 30 is associated with a different (unique) carrier frequency. In such embodiments, each optical signal having a different (unique) carrier frequency enables the processor 26 to control the optical transmitters 28 to simultaneously transmission optical signals in a common region (area) of the user's skin as the processor 26 may be configured to determine the optical transmitter 28 from which each optical signal received by an optical transmitter 30 originated and the signal path along which that optical signal traveled to an optical receiver 30.

In other embodiments, processor 26 may control the modulators 32 and the optical transmitters to utilize fewer modulated electronic signals each have a different (unique) carrier frequency than the number of optical transmitters 28. For instance, in embodiments, processor 26 may separate a plurality of optical transmitters 28 on the bottom wall of the electronic wearable device 10 into regions and control the modulators 32 and the optical transmitters to cause all optical signals traveling within or originating from a region to utilize or be associated with a different carrier frequency than other optical signals traveling within or originating from that region. For example, the bottom wall of the electronic wearable device 10 may be separated into three regions and processor 26 may control the modulators 32 and the optical transmitters to cause all optical signals traveling within or originating from a first region to be associated with a different carrier frequency than other optical signals traveling within or originating from the first region, all optical signals traveling within or originating from a second region to be associated with a different carrier frequency than other optical signals traveling within or originating from the second region, and all optical signals traveling within or originating from a third region to be associated with a different carrier frequency than other optical signals traveling within or originating from the third region. At any moment in time, one or more optical transmitters 28 in a region may output optical signals having a carrier frequency that is the same as a carrier frequency of one or more optical transmitters 28 in the other two regions.

In other embodiments, processor 26 may control optical transmitters 28 to output optical signals in repeating cycles of optical signal transmissions by dividing a cycle period into transmissions periods for a plurality of optical transmitters 28 that are each allocated one of a plurality of predetermined a transmission period and all of the optical signals output within a given transmission period may be associated with a different carrier frequency than other optical signals output during that transmission period. For example, a transmission cycle of optical signals by a plurality of optical transmitters within a 300 microsecond cycle period may be separated into three transmission periods of 100 microseconds each and all optical signals output by an optical transmitter 28 within a first time period (start of each cycle period to 100 microseconds) may be associated with a different carrier frequency than other optical signals output during that first time period, all optical signals output by an optical transmitter 28 within a second time period (100 microseconds to 200 microseconds) may be associated with a different carrier frequency than other optical signals output during that second time period, and all optical signals output by an optical transmitter 28 within a third time period (200 microseconds to 300 microseconds) may be associated with a different carrier frequency than other optical signals output during that third time period. For any time period, one or more optical transmitters 28 may output optical signals having a carrier frequency that is the same as a carrier frequency of one or more optical transmitters 28 in the other two time periods.

Returning to FIG. 9A, the bottom wall of the electronic wearable device 10 includes a plurality of optical receivers 28 (RX1-RXn), a plurality of optical transmitter arrays TXA1(A)-TXAn(A) and a plurality of optical transmitter arrays TXA1(B)-TXAn(B). The plurality of optical transmitter array TXA1(A)-TXAn(A) are separated from a proximate optical receiver 28 (RX1-RXn) by a distance that is a first distance and the plurality of optical transmitter arrays TXA1(B)-TXAn(B) are separated from an optical receiver 28 (RX1-RXn) by a second distance, the second distance being shorter than the first distance. For the configuration shown in FIG. 9A, the plurality of optical receivers 28 (RX1-RXn) are closer to one of the plurality of optical transmitter arrays TXA1(B)-TXAn(B) than the plurality of optical transmitter array TXA1(A)-TXAn(A).

As shown in FIG. 9B, each optical transmitter array may include a plurality of optical transmitters 28 that are located at a substantially common position on the bottom wall of the electronic wearable device 10 and each optical transmitter 28 outputs an optical signal associated with a predetermined carrier frequency that travels along a signal path to an optical receiver 30. For example, an optical transmitter array may include four optical transmitters 28 that each output optical signals associated with a predetermined carrier frequency (f₁-f₄) and a predetermined wavelength (λ). The location of each optical transmitter 28 within an optical transmitter array may be specified using (x,y) coordinates, as shown in FIG. 9D, in which the four optical transmitters 28 in optical transmitter array TXA1(A) are specified as being positioned at one of the four locations depicted in FIG. 9B. Specifically, a first optical transmitter 28 of optical transmitter array TXA1(A) may be in a lower, left position (0, 0), have a predetermined carrier frequency (f₁) and a predetermined wavelength (λ) between 610 nm and 640 nm that is visibly perceived as the color red. Similarly, a second optical transmitter 28 of optical transmitter array TXA1(A) may be in a lower, right position (0, 1), have a predetermined carrier frequency (f₂) and a predetermined wavelength (λ) between 640 nm and 670 nm that is visibly perceived as the color red. Similarly, a third optical transmitter 28 of optical transmitter array TXA1(A) may be in an upper, left position (1, 0), have a predetermined carrier frequency (f₃) and a predetermined wavelength (λ) between 670 nm and 720 nm that is visibly perceived as the color red. Similarly, a fourth optical transmitter 28 of optical transmitter array TXA1(A) may be in an upper, right position (1, 1), have a predetermined carrier frequency (f₄) and a predetermined wavelength (λ) between 920 nm and 950 nm that is in the infrared band. In embodiments, an optical transmitter array presented in FIG. 9A may consist of or be replaced with a single optical transmitter 28, which may be positioned at a center position of where the optical transmitter array would otherwise be positioned. For example, in the embodiment described in FIG. 9D, an optical transmitter array TXA1(B) may consist of a single optical transmitter 28 that outputs optical signals having a predetermined carrier frequency (f₅) and a predetermined wavelength (λ) between 520 nm and 550 nm that is visibly perceived as the color green. The position, carrier frequency and wavelength of each optical transmitter 28 of optical transmitter array TXA1(A) and optical transmitter array TXA1(B), as well as two signals to proximate optical receivers 30 (RX1 and RX2), is provided in FIG. 9D.

Similarly, in embodiments, five additional carrier signals each having different frequencies (f₆-f₁₀) would be associated with the optical transmitters 28 within optical transmitter arrays TXA2(A) and TXA2(B). Specifically, a first optical transmitter 28 of optical transmitter array TXA2(A) may be in a lower, left position (0, 0), have a predetermined carrier frequency (f₆) and a predetermined wavelength (λ) between 610 nm and 640 nm that is visibly perceived as the color red. Similarly, a second optical transmitter 28 of optical transmitter array TXA2(A) may be in a lower, right position (0, 1), have a predetermined carrier frequency (f₇) and a predetermined wavelength (λ) between 640 nm and 670 nm that is visibly perceived as the color red. Similarly, a third optical transmitter 28 of optical transmitter array TXA2(A) may be in an upper, left position (1, 0), have a predetermined carrier frequency (f₈) and a predetermined wavelength (λ) between 670 nm and 720 nm that is visibly perceived as the color red. Similarly, a fourth optical transmitter 28 of optical transmitter array TXA2(A) may be in an upper, right position (1, 1), have a predetermined carrier frequency (f₉) and a predetermined wavelength (λ) between 920 nm and 950 nm that is in the infrared band. An optical transmitter array TXA2(B) may consist of a single optical transmitter 28 that outputs optical signals having a predetermined carrier frequency (f₁₀) and a predetermined wavelength (λ) between 520 nm and 550 nm that is visibly perceived as the color green.

Extending such a configuration to other optical transmitter arrays depicted in FIG. 9A, in embodiments, yet another five additional carrier signals each having different frequencies (fₙ-fₙ₊₄) would be associated with the optical transmitters 28 within transmitters TXA2(A) and TXA2(B). Specifically, a first optical transmitter 28 of optical transmitter array TXAn(A) may be in a lower, left position (0, 0), have a predetermined carrier frequency (fₙ) and a predetermined wavelength (λ) between 610 nm and 640 nm that is visibly perceived as the color red. Similarly, a second optical transmitter 28 of optical transmitter array TXAn(A) may be In a lower, right position (0, 1), have a predetermined carrier frequency (fₙ₊₁) and a predetermined wavelength (λ) between 640 nm and 670 nm that is visibly perceived as the color red. Similarly, a third optical transmitter 28 of optical transmitter array TXAn(A) may be in an upper, left position (1, 0), have a predetermined carrier frequency (fₙ₊₂) and a predetermined wavelength (λ) between 670 nm and 720 nm that is visibly perceived as the color red. Similarly, a fourth optical transmitter 28 of optical transmitter array TXAn(A) may be in an upper, right position (1, 1), have a predetermined carrier frequency (fₙ₊₃) and a predetermined wavelength (λ) between 920 nm and 950 nm that is in the infrared band. An optical transmitter array TXAn(B) may consist of a single optical transmitter 28 that outputs optical signals having a predetermined carrier frequency (fₙ₊₄) and a predetermined wavelength (λ) between 520 nm and 550 nm that is visibly perceived as the color green.

It is to be understood that each modulator 32 may include circuitry such as signal multipliers and signal dividers to generate each of the modulated electronic signals having a different frequency.

As shown in FIG. 9A, the optical signal output by each optical transmitter 28 of the optical transmitter array TXA1(A) passes substantially along a common signal path to proximate optical receivers 30, such as signal path P1A-1 to a first optical receiver 30 (RX1), signal path P1A-2 to a second optical receiver 30 (RX2) and signal path P1A-n to a proximate optical receiver 30 (RXn).

Processor 26 may cause a plurality of optical transmitters 28 to output optical signals having specific wavelengths to determine physiological information about the user. For instance, it is generally understood that the absorption of the optical signal by a typical user's blood varies according to a wavelength of the optical signal and there are differences in the absorption of optical signal between oxygenated blood and deoxygenated blood. A plot of an absorption coefficient versus optical signal wavelength is shown in FIG. 9C and illustrates the variability of the absorption according to wavelength as well as the differences in absorption between oxygenated blood and deoxygenated blood. The wavelengths at which the absorption coefficient for oxygenated blood absorption of the optical signal is maximized result in a PPG signal with the cardiac component having an improved signal-to-noise ratio, leading to accurate heart rate determination, than a PPG signal generated from optical signals having other wavelengths. In embodiments, the processor 26 may cause each of a plurality of optical transmitters 28 to output an optical signal at a wavelength in the portion of the spectrum labeled in FIG. 9C as “heart rate” the reflections of which may be used by an optical receiver to generate a PPG signal that is utilized by the processor 26 to determine a user's heart rate. The wavelengths of the two (or, in embodiments, three) optical signals resulting in at least two PPG signals the processor 26 is configured to use to determine the pulse oximetry of the user. In order to differentiate the response of the optical signal due to the oxygenated blood from the response due to the deoxygenated blood, processor 26 may cause optical transmitters 28 to output an optical signal for two wavelengths in regions of the plot of FIG. 9C where there is greater separation between the absorption coefficients of the oxygenated blood and the deoxygenated blood. For instance, processor 26 may cause a plurality of optical transmitters 28 to output optical signals at a wavelength (λ) at which the separation between the oxygenated and deoxygenated blood absorption coefficients in the red wavelengths of the visible spectrum (TX1, TX2 and TX3) and in wavelengths of the lower infrared spectrum (TX4) is sufficient for processor 26 to determine pulse oximetry and other physiological information about the user.

For example, in embodiments, the plurality of optical transmitter arrays TXA1(A)-TXAn(A) positioned on the bottom wall of the electronic wearable device 10 may output optical signals of a wavelength that may enable processor 26 to determine physiological information about the user. For example, in the embodiment shown in FIG. 9C, optical transmitter arrays TXA1(B)-TXAn(B) may each include or consist of an optical transmitter 28 that outputs optical signals having a predetermined carrier frequency (f₅) and a wavelength (λ) between 520 nm and 550 nm that are received by an optical receiver 30 as one of a plurality of optical signals in a broadband electronic signal that processor 26 may isolate and utilize to determine a heart rate for the user. Similarly, optical transmitter arrays TXA1(A)-TXAn(A) may each include a first optical transmitter 28 that outputs optical signals having a predetermined carrier frequency (f₁) and a wavelength (λ) between 610 nm and 640 nm, a second optical transmitter 28 that outputs optical signals having a predetermined carrier frequency (f₂) and a wavelength (λ) between 640 nm and 670 nm, a third optical transmitter 28 that outputs optical signals having a predetermined carrier frequency (f₃) and a wavelength (λ) between 690 nm and 720 nm, a fourth optical transmitter 28 that outputs optical signals having a predetermined carrier frequency (f₄) and a wavelength (λ) between 920 nm and 950 nm, that are each received by an optical receiver 30 as one of a plurality of optical signals in a broadband electronic signal and utilized by processor 26 to determine physiological information, such as pulse oximetry level (also known as a level of blood oxygen saturation, or SpO2), an estimated stress level, a maximum rate of oxygen consumption (VO2 max), or the like, for the user.

The techniques disclosed herein enable a processor 26 to determine, for each PPG signal, a source of the optical signal and the signal path from the optical transmitter 28 to the optical receiver 30 through which the optical signal passed in the user's skin and select one or more PPG signals based on the determined signal path and a signal to noise ratio to be utilized with determining physiological information for the user. As shown in FIG. 9A, a first PPG signal may be associated with an optical signal that passed along signal path “P1A-1” extending from an optical transmitter 28 of optical transmitter array TXA1(A) to a first optical receiver 28 (RX1) and a second PPG signal may be associated with an optical signal that passed along signal path “P1B-1” extending from an optical transmitter 28 of optical transmitter array TXA1(B) to the first optical receiver 28 (RX1). Similarly, a third PPG signal may be associated with an optical signal that passed along signal path “P2A-2” extending from an optical transmitter 28 of optical transmitter array TXA2(A) to second optical receiver 28 (RX2) and a fourth PPG signal may be associated with an optical signal that passed along signal path “P2B-2” extending from an optical transmitter 28 of optical transmitter array TXA2(B) to the second optical receiver 28 (RX2).

It is to be understood that each optical signal output into and reflected from the user's skin may be received by any optical receiver 30 that is sufficiently proximate to the optical transmitter 28 that output the optical signal. For instance, exemplary signal paths “P1A-2” and “P2A-1” are shown for the optical signals passing from an optical transmitter 28 of optical transmitter TXA1(A) to the second optical receiver 30 (RX2) and the optical signals passing from TXA2(A) to the first optical receiver 30 (RX1), respectively. As the optical signals output by an optical transmitter 28 of the optical transmitter array TXA1(A) and an optical transmitter 28 of the optical transmitter array TXA2(A) travel over signal paths “P1A-2” and “PA2-1,” respectively, both the first optical receiver (RX1) and the second optical receiver (RX2) would receive those optical signals from the user's skin within a broadband electronic signal simultaneously and the resulting PPG signals utilized by processor 26 would correspond in time. Similarly, exemplary signal paths “P1B-2” and “P2B-1” are shown for the optical signals passing from an optical transmitter 28 of optical transmitter TXA1(B) to the second optical receiver 30 (RX2) and the optical signals passing from TXA2(B) to the first optical receiver 30 (RX1), respectively. As the optical signals output by an optical transmitter 28 of the optical transmitter array TXA1(B) and an optical transmitter 28 of the optical transmitter array TXA2(B) travel over signal paths “P1B-2” and “P2B-1,” respectively, both the first optical receiver (RX1) and the second optical receiver (RX2) would receive those optical signals from the user's skin within a broadband electronic signal simultaneously and the resulting PPG signals utilized by processor 26 would correspond in time.

It is to be understood that similar arrangements of optical components may exist, so additional signal paths are illustrated for “N” number of such optical component groups. In other words, an additional PPG signal would also be associated with an optical signal that passed along signal path “PnA-n” extending from an optical transmitter 28 of optical transmitter array TXAn(A), which corresponds to a transmitter array location and wavelength, to a proximate optical receiver 28 (RXn) and another PPG signal would also be associated with an optical signal that passed along signal path “PnB-n” extending from an optical transmitter 28 of optical transmitter array TXAn(B) to the proximate optical receiver 28 (RXn). Exemplary signal paths “PnA-1” and “PnB-1” are shown for the optical signals passing from an optical transmitter 28 of optical transmitter TXAn(A) to the first optical receiver 30 (RX1) and the optical signals passing from TXAn(B) to the first optical receiver 30 (RX1), respectively. As the optical signals output by an optical transmitter 28 of the optical transmitter array TXAn(A) and an optical transmitter 28 of the optical transmitter array TXAn(B) travel over signal paths “PnA-1” and “PnB-1,” respectively, the first optical receiver (RX1) would receive those optical signals from the user's skin within a broadband electronic signal simultaneously and the resulting PPG signals utilized by processor 26 would correspond in time. Similarly, exemplary signal paths “PnA-2” and “PnB-2” are shown for the optical signals passing from an optical transmitter 28 of optical transmitter TXAn(A) to the second optical receiver 30 (RX2) and the optical signals passing from TXAn(B) to the second optical receiver 30 (RX2), respectively. As the optical signals output by an optical transmitter 28 of the optical transmitter array TXAn(A) and an optical transmitter 28 of the optical transmitter array TXAn(B) travel over signal paths “PnA-2” and “PnB-2,” respectively, the second optical receiver (RX2) would receive those optical signals from the user's skin within a broadband electronic signal simultaneously and the resulting PPG signals utilized by processor 26 would correspond in time. Similarly, exemplary signal paths “P1A-n,” “P2a-n,” “P1B-n” and “P2B-n” are shown for the optical signals passing from an optical transmitter 28 of optical transmitters TXA1(A), TXA2(A), TXA1(B) and TXA2(B), respectively, to the optical receiver 28 (RXn). As the optical signals output by an optical transmitter 28 of the optical transmitter arrays TXA1(A), TXA2(A), TXA1(B) and TXA2(B) travel over signal paths “P1A-n,” “P2a-n,” “P1B-n” and “P2B-n,” respectively, the optical receiver 28 (RXn) would receive those optical signals from the user's skin within a broadband electronic signal simultaneously and the resulting PPG signals utilized by processor 26 would correspond in time.

As an optical receiver 28 may receive optical signals that were simultaneously output by optical transmitters 28 at different locations, the multiplexing of optical signals transmitted by the optical transmitters 28 enables processor 26 to determine and sample different signal paths through which each optical signal traveled to an optical receiver 30 from different optical transmitters 28. For example, processor 26 may determine physiological information using PPG signals associated with optical signals output by the first optical receiver (RX1) by selecting one or more of signal path “P1A-1” (associated with optical transmitter TXA1(A)), signal path “P1B-1” (associated with optical transmitter TXA1(B)) and signal path “P2A-1” (associated with optical transmitter TXA2(A)).

Similar to the exemplary wavelengths and five carrier frequencies (f₁-f₅) of the optical transmitters 28 of optical transmitter arrays TXA1(A) and TXA1(B), five additional carrier signals each having different frequencies (f₆-f₁₀) are associated with the optical transmitters 28 of optical transmitter arrays TXA2(A) and TXA2(B). By extension, for “N” such groups of optical transmitter arrays, five more carrier signals each having different frequencies (fₙ-fₙ₊₄) would be utilized for each group until all optical transmitters 28 of “N” optical transmitter arrays is each associated with a carrier signal having a different frequency.

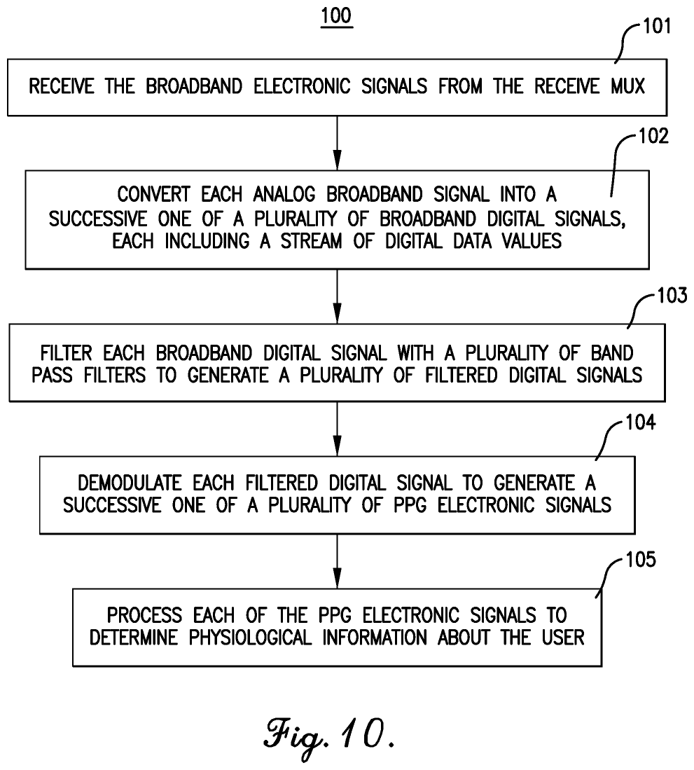

In embodiments, the processor 26 and/or each receive processor circuit 36 may execute, process, or run instructions, code or software to perform the process steps shown in FIG. 10. FIG. 10 is a flowchart 100 for determining for filtering and demodulating optical signals received from the user's skin to generate PPG electronic signals in accordance with embodiments of the technology in accordance with embodiments of the technology. In various embodiments, one or more regions of method 100 (or the entire method 100) may be implemented by any suitable device. For example, one or more portions of method 100 may be performed by processor 26 in conjunction with one or more receive processor circuits 36, as shown in FIG. 6B. Method 100 represents the calculations performed to calculate and display physiological information about the user using broadband electronic signals output by the optical receiver 30 based on the reflections of a plurality of optical signals each having different carrier frequency that are received from the user's skin. For example, method 100 may represent the iterative steps taken to generate PPG electronic signals by demodulating filtered digital broadband electronic signals and calculate physiological information about the user, as discussed herein with reference to FIGS. 6A-6B, with the calculated physiological information being displayed via display 16.

Referring to process step 101, the processor 26 receives analog broadband electronic signals from a receive multiplexer 34 (block 101). The receive multiplexer 34 may redirect analog broadband electronic signals the receive multiplexer 34 received from an optical receiver 30. Alternatively, the processor 26 may receive the broadband electronic signals from the optical receivers 30 directly-that is, the processor 26 may receive the first broadband electronic signal from the first optical receiver 30 and the second broadband electronic signal from the second optical receiver 30. This may also include, for example, receiving broadband electronic signals from an optical receiver 30, as discussed with reference to FIG. 6B. In other embodiments, if an analog to digital converter, such as the ADC 42, is not integrated within the processor 26, then the broadband electronic signals are received by the ADC 42.

Referring to process step 102, each broadband electronic signal (in its analog form output

by the optical receiver 30 or the receiver multiplexer 34) is converted to a successive one of a plurality of broadband digital signals, each including a stream of digital data values. That is, the first broadband electronic signal is converted to a first broadband digital signal, and the second broadband electronic signal is converted to a second broadband digital signal. The conversion may include electronic waveform sampling of each broadband electronic signal, such that the result of the conversion is that each broadband digital signal includes a stream of digital data values, with each digital data value representing the voltage or current as it was sampled. The conversion may be performed by an analog to digital converter integrated within the processor 26 or by one or more ADCs 42. As the digital broadband signals may be generated from analog broadband signals by an ADC 42 within processor 26 or an ADC 42 that is external to processor 26, but controlled by processor 26, the processor 26 and/or each receive processor circuit 36 may convert each broadband electronic signal to a successive one of a plurality of broadband digital signals. The analog broadband signals may be output by an optical receiver 30 or a receive multiplexer 34 before an ADC 42 receives the analog broadband signal and converts the analog optical signals to digital data values, or streams of digital data values of the optical signals.

Referring to process step 103, each broadband digital signal is filtered with a plurality of band pass filters to generate a filtered digital signal that is includes one or more frequency components of the broadband electronic signal that have a frequency value substantially equal to the carrier frequency. Each band pass filter may be implemented as a finite impulse response (FIR) filter, infinite impulse response (IIR) filter, or other DSP process. A successive band pass filter may be implemented for each carrier frequency that is utilized by the modulators 32. In addition, each band pass filter passes portions of the broadband digital signal corresponding to a selected carrier frequency and filters out portions of the broadband digital signal corresponding to the other carrier frequencies. Each band pass filter outputs or generates a filtered digital signal for each broadband digital signal that it operates on. For example, the first broadband digital signal (converted from the first broadband electronic signal output by the first optical receiver 30) is band pass filtered multiple times, one time for each of the carrier frequencies, with each band pass filtering process generating a successive one of the filtered digital signals derived from the first broadband signal, the second broadband digital signal is filtered multiple times, one time for each of the carrier frequencies, with each band pass filtering process generating a successive one of the filtered digital signals derived from the second broadband signal, and so forth, if more than two optical receivers are utilized. Accordingly, in embodiments, the processor 26 and/or each receive processor circuit 36 may receive the broadband electronic signal, perform a band pass function based on a predetermined carrier frequency, and output a first filtered electronic signal having the first carrier frequency.

Referring to process step 104, each filtered digital signal is demodulated to generate and output a successive one of a plurality of PPG electronic signals. The demodulation process implemented by the processor 26 and/or each receive processor circuit 36 extracts the amplitude of the carrier frequency component from each filtered digital signal which leaves a digital signal without the carrier frequency while retaining the effects that the human tissue had on the optical signal. For example, the demodulation process demodulates the first filtered digital signal from the first broadband signal to generate a first PPG electronic signal, demodulates the second filtered digital signal from the first broadband signal to generate a second PPG electronic signal, and so forth, demodulating each filtered digital signal from each broadband signal. The demodulated filtered optical signal is a PPG electronic signal that includes the characteristics imparted on the optical signal by traveling through the user's skin.

Referring to process step 105, the processor 26 then utilizes each of the PPG electronic signals, after the amplitude of carrier frequency has been extracted, to determine physiological information about the user, such as a user's heart rate, pulse oximetry level (also known as a level of blood oxygen saturation, or SpO2), an estimated stress level, a maximum rate of oxygen consumption (VO2 max), or the like.

The circuitry shown in FIGS. 6A and 6B and described above depicts just one configuration of PPG electronic signal processing wherein there are equal numbers of modulators 32, optical transmitters 28, optical receivers 30, and receive processor circuits 36. In other embodiments, there may be unequal numbers of the components. For example, there may be fewer modulators 32 than optical transmitters 28 such that at least one modulator 32 may provide the modulated electronic signal (with a particular carrier frequency) to more than one optical transmitter 28. There may be differing numbers of optical transmitters 28 and optical receivers 30, as well as differing numbers of optical receivers 30 and receive processor circuits 36.

It is to be understood that, in embodiments, processor 26 may control the plurality of modulators 32 to implement a modulation function that results in the output of a modulated electronic signal having a predetermined frequency, phase and/or waveform that enables each optical signal, and each optical transmitter 28 from which the optical signal was output, to be distinguished from optical signals output by other optical transmitters 28.

For instance, in embodiments, processor 26 may control the plurality of modulators 32 to each output a modulated electronic signal having a different phase than other modulated electronic signals. For example, processor 26 may control a first modulator 32 to output a first modulated electronic signal having a first signal phase and a second modulated electronic signal having a second signal phase. The first modulated electronic signal and the second modulated electronic signal may have the same frequency and amplitude. The processor 26 may select the first signal phase and the second signal phase to have a phase offset of 180 degrees, which may be a sufficient phase difference between the two modulated electronic signals that may enable the processor 26 to reliably identify and separate optical signals associated with those modulated electronic signals with accuracy. As another example, processor 26 may control a first modulator 32 to output a first modulated electronic signal having a first signal phase, a second modulated electronic signal having a second signal phase, a third modulated electronic signal having a third signal phase, and a fourth modulated electronic signal having a fourth signal phase. The processor 26 may select the first signal phase, the second signal phase, the third signal phase and the fourth signal phase to have a phase offset of 90 degrees (e.g., 0 degrees, 90 degrees, 180 degrees, 270 degrees for the first signal phase, the second signal phase, the third signal phase and the fourth signal phase, respectively), which may be a sufficient phase difference between the four modulated electronic signals that may enable the processor 26 to reliably identify and separate those modulated electronic signals with accuracy.

In embodiments, processor 26 may control the plurality of modulators 32 to each output a modulated electronic signal having a different waveform than other modulated electronic signals. For example, processor 26 may control a first modulator 32 to output a first modulated electronic signal having a first waveform and a second modulated electronic signal having a second waveform. The first modulated electronic signal and the second modulated electronic signal may have the same frequency and amplitude. The processor 26 may select the first waveform and the second waveform that are sufficiently distinct to enable the processor 26 reliably identify and separate those optical signals associated with modulated electronic signals with accuracy.

Throughout this specification, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either present technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112 (f) unless traditional means-plus-function language is expressly recited, such as “means for” or “step for” language being explicitly recited in the claim(s).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

What is claimed is:

1. An electronic wearable device comprising:
a housing including a bottom wall configured to contact a user's skin;
a first modulator configured to output a first modulated electronic signal having a first carrier frequency;
a second modulator configured to output a second modulated electronic signal having a second carrier frequency;
a first optical transmitter coupled with the first modulator, positioned on the bottom wall and configured to output a first optical signal based on the first modulated electronic signal, the first optical signal output into the user's skin and having the first carrier frequency;
a second optical transmitter coupled with the second modulator, positioned on the bottom wall and configured to output a second optical signal based on the second modulated electronic signal, the second optical signal output into the user's skin and having the second carrier frequency;
an optical receiver positioned on the bottom wall and configured to receive the first optical signal and the second optical signal from the user's skin as a broadband electronic signal including the first optical signal and the second optical signal associated with the first carrier frequency and the second carrier frequency, respectively, and output the broadband electronic signal;
a first receive processor circuit configured to receive the broadband electronic signal including the first optical signal having the first carrier frequency and output a first photoplethysmogram (PPG) electronic signal corresponding to the first optical signal based on the first carrier frequency; and
a second receive processor circuit configured to receive the broadband electronic signal including the second optical signal having the second carrier frequency and output a second PPG electronic signal corresponding to the second optical signal based on the second carrier frequency.

2. The electronic wearable device of claim 1, wherein the broadband electronic signal includes the first carrier frequency and the second carrier frequency as frequency components.

3. The electronic wearable device of claim 2, wherein the first receive processor circuit is electrically coupled with the optical receiver and includes a first filter configured to receive the broadband electronic signal, perform a band pass function based on the first carrier frequency, and output a first filtered electronic signal having the first carrier frequency.

4. The electronic wearable device of claim 3, wherein the first receive processor circuit includes a first demodulator configured to receive the first filtered electronic signal, extract the amplitude of the first carrier frequency, and output the first PPG electronic signal.

5. The electronic wearable device of claim 2, wherein the second receive processor circuit is electrically coupled with the optical receiver and includes a second band pass filter configured to receive the broadband electronic signal, perform a band pass function based on the second carrier frequency, and output a second filtered electronic signal having the second carrier frequency.

6. The electronic wearable device of claim 5, wherein the second receive processor circuit includes a second demodulator configured to receive the second filtered electronic signal, extract the amplitude of the second carrier frequency, and output the second PPG electronic signal.

7. The electronic wearable device of claim 1, wherein the first modulator and the second modulator each include a transconductance amplifier, wherein the first optical transmitter and the second optical transmitter are each a light-emitting diode (LED), and wherein the optical receiver is a photodiode.

8. The electronic wearable device of claim 1, wherein the first optical transmitter and the second optical transmitter are positioned at a first location on the bottom wall, and wherein the optical receiver is positioned at a second location on the bottom wall.

9. The electronic wearable device of claim 1, wherein the first receive processor circuit includes a first analog to digital converter configured to receive the first PPG electronic signal and output the first PPG electronic signal as a stream of digital data values that represent a PPG waveform, and wherein the second receive processor circuit includes a second analog to digital converter configured to receive the second PPG electronic signal and output the second PPG electronic signal as a stream of digital data values that represent a PPG waveform.

10. The electronic wearable device of claim 1, wherein the first optical transmitter is positioned at a first location on the bottom wall, wherein the second optical transmitter is positioned at a second location on the bottom wall, and wherein the optical receiver is positioned at a third location on the bottom wall.

11. An electronic wearable device comprising:
a housing including a bottom wall configured to contact a user's skin;
a plurality of modulators, each modulator configured to output one of a plurality of modulated electronic signals, each modulated electronic signal having a predetermined carrier frequency;
a plurality of optical transmitters positioned on the bottom wall and configured to output a plurality of optical signals, each optical transmitter coupled with one of the plurality of modulators and configured to receive one of the modulated electronic signals and output an optical signal into a user's skin that corresponds to the carrier frequency of the modulated electronic signal;
an optical receiver positioned on the bottom wall and configured to receive the plurality of optical signals from the user's skin as a broadband electronic signal including optical signals associated with at least two carrier frequencies, and output the broadband electronic signal; and
a plurality of receive processor circuits, each receive processor circuit and configured to receive the broadband electronic signal and output one of a plurality of photoplethysmogram (PPG) electronic signals corresponding to the one of the plurality of optical signals.

12. The electronic wearable device of claim 11, wherein each receive processor circuit is electrically coupled with the optical receiver and includes:
a filter configured to output a filtered electronic signal corresponding to one of the plurality of carrier frequencies, and
a demodulator configured to receive the filtered electronic signal and output one of the plurality of PPG electronic signals.

13. The electronic wearable device of claim 12, wherein at least two of the plurality of optical transmitters are positioned at a first location on the bottom wall, and wherein the optical receiver is positioned at a second location on the bottom wall.

14. The electronic wearable device of claim 11, wherein a first of the plurality of optical transmitters is positioned at a first location on the bottom wall, wherein a second of the plurality of optical transmitters is positioned at a second location on the bottom wall, and wherein the optical receiver is positioned at a third location on the bottom wall.

15. The electronic wearable device of claim 11, wherein each modulator includes a transconductance amplifier, wherein each of the plurality of optical transmitter is a light-emitting diode (LED), and wherein the of the plurality of optical receivers is a photodiode configured to receive the optical signals and output the broadband electronic signal having frequencies that include the carrier frequencies.

16. An electronic wearable device comprising:
a housing including a bottom wall configured to contact a user's skin;
a first modulator configured to output a first modulated electronic signal having a first carrier frequency;
a second modulator configured to output a second modulated electronic signal having a second carrier frequency;
a first optical transmitter coupled with the first modulator, positioned on the bottom wall and configured to output a first optical signal based on the first modulated electronic signal, the first optical signal output into the user's skin and having the first carrier frequency;
a second optical transmitter coupled with the second modulator, positioned on the bottom wall and configured to output a second optical signal based on the second modulated electronic signal, the second optical signal output into the user's skin and having the second carrier frequency;
an optical receiver positioned on the bottom wall and configured to receive the first optical signal and the second optical signal from the user's skin as an analog broadband electronic signal including the first optical signal and the second optical signal associated with the first carrier frequency and the second carrier frequency, respectively, and output the analog broadband electronic signal;
an analog to digital converter configured to receive the analog broadband electronic signal and output the broadband electronic signal as a stream of digital data values corresponding to the first optical signal and the second optical signal;
a memory element configured to store the first carrier frequency as corresponding to the first optical signal and the second carrier frequency as corresponding to the second optical signal; and
a processor coupled with the analog to digital converter and the memory element, the processor configured to:
receive the digital broadband electronic signal having the first carrier frequency components and the second carrier frequency components,
generate a first filtered electronic signal and a second filtered electronic signal having the first carrier frequency and the second carrier frequency, respectively,
demodulate the first filtered electronic signal and the second filtered electronic signal by extracting the amplitude of the first carrier frequency and the second carrier frequency, respectively, and
output a first PPG electronic signal and a second PPG electronic signal corresponding to the demodulated first filtered electronic signal and the demodulated second filtered electronic signal, respectively.

17. The electronic wearable device of claim 16, wherein the broadband electronic signal includes the first carrier frequency and the second carrier frequency as frequency components.

18. The electronic wearable device of claim 16, wherein the first modulator and the second modulator each include a transconductance amplifier, wherein the first optical transmitter and the second optical transmitter are each a light-emitting diode (LED), and wherein the optical receiver is a photodiode.

19. The electronic wearable device of claim 16, wherein the first optical transmitter and the second optical transmitter are positioned at a first location on the bottom wall, and wherein the optical receiver is positioned at a second location on the bottom wall.

20. The electronic wearable device of claim 16, wherein the first optical transmitter is positioned at a first location on the bottom wall, wherein the second optical transmitter is positioned at a second location on the bottom wall, and wherein the optical receiver is positioned at a third location on the bottom wall.
